(19) United States
(12) Patent Application Publication
(10) Pub. No.: US 2010/0178273 A1
(43) Pub. Date: Jul. 15, 2010

(54) METHODS AND COMPOSITIONS FOR TREATING MUCOSITIS

(75) Inventor: Pieter Rottiers, De Pinte (BE)

Correspondence Address: CASTELLANO PLLC, P.O. Box 1555, Great Falls, VA, 22066, US

(73) Assignee: ACTOGENIX NV CORPORATION

(21) Appl. No.: 12/665,355

(22) PCT Filed: Jun. 19, 2008
(86) PCT No.: PCT/EP08/04937

§ 371 Date: Jan. 25, 2010

(30) Foreign Application Priority Data
Jun. 20, 2007 (EP) 07012081.1
Feb. 18, 2008 (EP) 08101723.8

(51) Int. Cl. A61K 35/74; C12N 1/21; A61P 1/04

(52) U.S. Cl. 424/93.2; 435/252.3

(57) ABSTRACT

The invention relates generally to the field of mucositis. More particularly, the present invention relates to methods and compositions for treating and preventing mucositis. In particular for treating or preventing oral mucositis, especially stomatitis and esophagitis. The invention generally relates to the use of recombinant non-pathogenic and non-invasive microorganisms, in particular recombinant bacterium or yeast expressing a trefoil peptide in particular in combination with one or more factors that target the mucus, tight junctions or extracellular matrix and/or factors typically used in the treatment of mucositis.

### FIELD OF THE INVENTION

The invention relates generally to the field of mucositis. More particularly, the present invention relates to methods and compositions for treating and preventing mucositis. In particular for treating or preventing oral, intestinal or rectal mucositis, especially stomatitis, proctitis and esophagitis. The invention generally relates to the use of recombinant non-pathogenic and non-invasive bacterium expressing a trefoil peptide in particular in combination with one or more factors that target the mucus, tight junctions or extracellular matrix and/or factors typically used in the treatment of mucositis.

### BACKGROUND TO THE INVENTION

One complication of radiation therapy (RT) and chemotherapy (CT) is the damage that occurs in the mucosal lining of the alimentary tract, especially to the oral, oropharyngeal intestinal and rectal mucosa. This damage is called mucositis and can lead to severe inflammation, lesions and ulcerations of the epithelia. Patients may experience intense pain, nausea and gastro-enteritis. They are highly susceptible to infections and the damage is a dose-limiting toxicity of cancer therapy and affects an important fraction of cancer patients world-wide.

Upon CT and/or RT, the oral cavity is one of the areas which is highly affected by the complications arising from such treatment; indeed, virtually all patients treated for tumours of the head and neck, and around 40% of those subjected to RT and/or CT for tumours in other locations (leukaemias or lymphomas) develop the aforementioned complications affecting the oral cavity and the rectum (Minerva Stomatol. 2002: 51:173-86).

In general, the term “mucositis” is understood to mean a clinical picture characterised by the presence of reduced epithelial thickness, intense erythema and ulcers, associated with a painful symptom complex and the possible occurrence of infection and hemorrhage (Oncologist 1998: 3:446-52; Oncologist 1999: 11:261-6).

In general, mucositis appears within 5 to 10 days of the drug or radiation treatment and can last several weeks. The severity of mucositis can limit subsequent doses of chemotherapy or radiation. Patients suffering from mucositis may need several weeks, or more, of intravenous feeding as a result of the mouth ulcers, cramps, extreme pain, gut denuding and severe diarrhea.

An even more serious consequence of mucositis is that the lesions can act as sites of secondary infections and portals of entry for endogenous oral microorganisms. Mucositis is therefore a significant risk factor for life-threatening systemic infection; the risk of systemic infection is exacerbated by concomitant neutropenia, which is another complication associated with chemotherapy. Patients with mucositis and neutropenia have a relative risk for a life-threatening systemic infection that is at least four times greater than that of individuals without mucositis.

The incidence and severity of mucosal radiation toxicity has increased with the use of accelerated fractionated schedules and concurrent radio-chemotherapy. Squamous non-keratinizing stratified epithelia in mouth, oropharynx and esophagus show a hierarchal structure that consists of stem cells and committed proliferative cells in the lower layers and non-proliferating functionally mature cells in the upper layers. Loss of the superficial cells is compensated by proliferation of clonogenic keratinocytes and maturation in deeper layers, resulting in a continuous renewal of the mucosae. Radiation at therapeutic doses causes cell kill in the proliferative compartment but leaves the non-proliferating compartment functionally intact. Physiological cell loss at the mucosal surface is also unaffected by radiotherapy and continues at its normal rate. Progressive mucosal hypoplasia and eventual loss of functional cells occurs. Exposure of submucosal tissues, infection and other stressors cause release of mediators of inflammation and pain. This mechanism is consistent with the clinical observation of a 7-10 day delayed progressive discomfort, pain and functional disturbances of the irradiated regions of the upper digestive tract.

Until recently, standard management options for mucositis involved the use of analgesics or were directed against inflammation and infection that exacerbate symptoms. In the first case, agents are used which are capable of reducing mucous absorption of the chemotherapy drugs (for example cryotherapy, allopurinol or pilocarpine etc.), agents which reduce the changes in epithelial proliferation (for example beta-carotene, glutamine or silver nitrate etc.) or anti-inflammatory and antimicrobial agents (for example, mesalazine and/or chlorhexidine).

In the second case, use is made of agents which protect the mucosa (for example, sodium bicarbonate), anaesthetic or analgesic agents (for example, lidocaine, morphine and the derivatives thereof etc.), agents which accelerate the healing process (for example, vitamin E, tretinoin, laser therapy etc.) or special diets and/or specific oral hygiene regimens.

An increased understanding of the underlying pathobiology of mucositis has led to the identification of a number of potential mechanistically based approaches that are now becoming available for clinical use. These approaches are primarily based on the use biologics such as anti-inflammatory cytokines and growth factors in the treatment of CT- and/or RT-induced mucositis. In this respect, Palifermin (Kepivance™, Amgen) an N-truncated form of recombinant human keratinocyte growth factor (KGF)-1, recently became the first agent to be approved as an intervention for oral mucositis in patients with hematologic malignancies undergoing hematopoietic stem cell transplant (HSCT). The restricted use of palifermin in a specific clinical setting, demonstrates one of the limitations related to the clinical success of biologics. Perhaps more than for other forms of treatment, the therapeutic application of biologics depends on the choice of formulation, timing, route of administration, dosing and stability. In addition to cytokine and growth factor receptors on normal tissues, these receptors may also be found on tumor tissue. There are accordingly concerns that the use of anti-inflammatory cytokines and growth factors may compromise the effects of chemotherapy and radiotherapy. For said reasons the labeled indication is restricted to cancers that do not express the particular growth factor, such as for example in the case of palifermin, the labeled indication is restricted to hematological cancers that do not express the KGF receptor. Therefore, it seems unlikely that palifermin would have any adverse effects on outcome in this type of malignancy.

Also the use of anti-inflammatory cytokines has certain drawbacks. For example, at low doses, the systemic administration of IL-10 will block the innate immune system and prevent bacterial clearing. At high doses it will induce a proinflammatory.

For IL-11 it is known that the subcutaneous administration has severe side effects with severe fluid retention and multi organ failure. In addition, IL-11 stimulates the proliferation of haematopoietic cells and stimulate platelet production.

In view of the above, further advances, such as further developments in the formulation of anti-inflammatory cytokines or growth factors are needed in the prevention or treatment of mucositis, in particular due to chemotherapy and/or radiotherapy, which remains a cause of suffering in patients suffering from tumors, such as in mucositis of the oral cavity in patients with head and/or neck tumors, and of the rectum (proctitis) in patients with rectal and prostate tumors.

### BRIEF DESCRIPTION OF THE DRAWINGS

FIG. 1 Relative Body Weight of C57BL/6 mice till day 7 in a model of 5-FU-induced mucositis.

Shows that mycTFF3+proteinF (#11) is different from pTREX+proteinF (#9) and mycTFF3+proteinF (#11) is different from pT1mycTFF2 (#6) at day 7 (P<0.05). No significant differences between other groups or at other time points. (One way anova and Bonferroni's multiple comparison test)

FIG. 2 Survival Curve of C57BL/6 mice in a model of 5-FU-induced mucositis, treated with LL expressing mTFF3 and Protein F.

FIG. 3 Weight Change: Area Under the Curve (AUC). The AUC, was calculated from the percent weight change exhibited by each animal in the study. This calculation was made using the trapezoidal rule transformation. Group means were calculated and are shown with error bars representing SEM for each group. A one way ANOVA was done to compare these groups. There were no statistically significant differences among the groups (P=0.055).

FIG. 4 Mean Daily Mucositis Scores. Mean group mucositis scores were obtained every other day. Error bars represent the standard error of the mean (SEM).

FIG. 5 Duration of Severe Mucositis. Number of days with mucositis scores≧3. To examine the levels of clinically significant mucositis, as defined by presentation with open ulcers (score≧3), the total number of days in which an animal exhibited an elevated score was summed and expressed as a percentage of the total number of days scored for each group. Statistical significance of observed differences was calculated using chi-square analysis. Significant improvements are denoted with an asterisk.

FIG. 6: Mean percent weight change. Animals were weighed daily, the percent weight change from day 0 was calculated, and group means and standard errors of the mean (SEM) calculated for each day.

FIG. 7: Weight Change: Area Under the Curve.

The area under the curve (AUC) was calculated for the percent weight change exhibited by each animal in the study. This calculation was made using the trapezoidal rule transformation. Group means were calculated and are shown with error bars representing SEM for each group. A One Way ANOVA was done to compare these groups. There were no statistically significant differences among the groups (P=0.119).

### DETAILED DESCRIPTION OF THE INVENTION

This invention relates to methods and compositions for treating and preventing mucositis, and is based on the finding that the use of recombinant non-pathogenic and non-invasive microorganisms, in particular recombinant bacterium and/or yeast, expressing a trefoil peptide optionally in combination with one or more factors that target the gastrointestinal tract epithelium, bring about a considerable improvement with regard to the mucosal damage of the mucosa in radiation/chemotherapy treated patients.

The members of the trefoil factor (TFF) family, which comprises the gastric peptides pS2, a.k.a. TFF1, the spasmolytic peptide SP, a.k.a. TFF2 and the intestinal trefoil factor ITF, a.k.a. TFF3, are abundantly secreted onto the mucosal surface by mucus-secreting cells of the gastrointestinal tract and characterized in that they are small (7-12 kDa) protease-resistant proteins that comprise a conserved distinct motif of six cysteine residues that define a so-called ‘trefoil’ domain.

As used herein, “trefoil peptide” is meant any polypeptide having at least a trefoil domain and retaining a biological activity characteristic of the naturally occurring trefoil peptides. Thus, preferred trefoil peptides may be any mammalian homolog or artificial polypeptide that are substantially identical, i.e. 70%, 75%, 80%, 85%, 87%, 89%, 90%, 92%, 93%, 94%, 96%, 98% or 99% identical to any one of human spasmolytic polypeptide (hSP; also known as TFF2, GenBank Accession No. NM-005423; SEQ ID NO:5), human pS2 (also known as TFF1, GenBank Accession No. XM-009779; SEQ ID NO: 3), human intestinal trefoil factor (hITF; also known as TFF3, SEQ ID NO: 1), and biologically active fragments thereof. If desired, the trefoil peptide may contain a cysteine residue outside of the trefoil domain suitable for disulfide bonding in the formation of homo- and heterodimers. Most preferably, the additional cysteine is C-terminal to the trefoil domain. Exemplary trefoil peptides include ITF 15-73, ITF 1-62, ITF 1-70, ITF 1-72, ITF 25-73, ITF 1-73 and ITF 21-73. In an further embodiment the peptides may be any mammalian homolog selected from the group consisting of human spasmolytic polypeptide (hSP; also known as TFF2, GenBank Accession No. NM-005423; SEQ ID NO:5), human pS2 (also known as TFF1, GenBank Accession No. XM-009779; SEQ ID NO: 3), and human intestinal trefoil factor (hITF; also known as TFF3, SEQ ID NO: 1). Preferably, a trefoil peptide is encoded by a nucleic acid molecule that hybridizes under high stringency conditions to the coding sequence of hITF (TFF3) (SEQ ID NO: 2), hSP (TFF2) (SEQ ID NO: 6), or hpS2 (TFF1) (SEQ ID NO: 4). Trefoil peptides amenable to methods of this invention may exist as monomers, dimers, or multimers. For example, trefoil peptide monomers may form an interchain disulfide linkage to form a dimer.

By “trefoil domain” is meant a polypeptide having a sequence substantially identical, i.e. 70%, 75%, 80%, 85%, 87%, 89%, 90%, 92%, 93%, 94%, 96%, 98% or 99% identical to any one of SEQ ID NOs: 7-10, which correspond to the trefoil domains of hpS2 30-70, hSP 30-71, hSP 80-20 and hITF 24-64, respectively, and retain at least one biologic activity characteristic of trefoil peptides. It is recognized in the art that one function of the six conserved cysteine residues is to impart the characteristic three-loop (trefoil) structure to the protein. The loop structure conforms to the general intrachain disulfide configuration of cys1-cys5 (corresponding to amino acid residues 25 and 51 of hITF; SEQ ID NO: 1), cys2-cys4 (corresponding to amino acid residues 35 and 50 of hITF; SEQ ID NO: 1), and cys3-cys6 (corresponding to amino acid residues 45 and 62 of hITF; SEQ ID NO: 1).

By “high stringency conditions” is meant any set of conditions that are characterized by high temperature and low ionic strength and allow hybridization comparable with those resulting from the use of a DNA probe of at least 40 nucleotides in length, in a buffer containing 0.5M NaHP04, pH 7.2, 7% SDS, 1 mM EDTA, and 1% BSA (Fraction V), at a′ temperature of 65° C., or a buffer containing 48% formamide, 4.8×SSC, 0.2 M Tris-C1, pH 7.6, 1×Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well known by those skilled in the art of molecular biology.

The percentage identity of polypeptide sequences can be calculated using commercially available algorithms which compare a reference sequence (e.g. SEQ ID NO:2, SEQ ID NO: 4 or SEQ ID NO:6 of the present invention) with a query sequence. Further details of assessing identity are described below.

“Mucositis” as used herein, refers to the destruction of mucosal epithelium of the alimentary tract including the lubricated inner lining of the mouth, nasal passages, vagina and urethra; in particular following chemotherapy or radiotherapy in patients suffering from tumors and to the symptomatic features associated therewith, i.e. pain, redness, inflammation, ulceration, or combinations thereof, affecting the mucosal epithelium.

Factors that target the alimentary tract epithelium, hereinafter also referred to as biologically active polypeptides for the treatment of mucositis, consist of molecules such as peptides, proteins, antibodies, glycoproteins or enzymes that either directly or indirectly ameliorate the symptomatic features associated with mucositis. Factors that directly ameliorate the symptomatic features associated with mucositis are factors, which are involved in the restitution of the gastrointestinal mucosa, i.e. the rapid migration of the surface epithelium over the basement membrane, the proliferation of said cells and their differentiation into the mucosal epithelium This group of factors primarily consists of regulatory peptide known to stimulate epithelial restitution and/or proliferation, such as for example the TFF family, the growth factors EGF, TGF-α, TGF-β, bFGF, HGF, IGF-I, IGF-II, R-spondin1, DKK1 and the cytokines IL-1β, IL-11, GM-CSF and IL-8; but also includes factors that stimulate cell migration such as motogens, integrins and metalloproteases.

Factors that indirectly ameliorate the symptomatic features associated with mucositis are factors, which prevent and/or reduce the damaging effect of the factors involved in the pathogenesis of mucositis, such as anti-oxidant agents. In the pathology of mucositis, early cellular damage is evident from;
the generation of free radicals (Reactive Oxigen Species (ROS));
the expression of early response genes, including c-jun, c-fos and Erg-I;
the activation of transcription factors such as nuclear factor kappa beta (NF-κ), the hSNK gene and vascular adhesion molecules;
the upregulation of pro-inflammatory cytokines, including tumor necrosis factor alpha, and the interleukins, in particular IL-1β and IL-6; and the release of a range of destructive proteins and molecules, such as nitric oxide (NO), ceramide and metalloproteinases (MMPs).

Hence in one embodiment of the present invention, factors that indirectly ameliorate the symptomatic features associated with mucositis are;
factors that prevent the damage due to free radicals, such as for example superoxide dismutases that detoxifies ROS, or Keratinocyte Growth Factor that activates NRF2 to induce superoxide dismutase;
anti-inflammatory factors including cytokines such as IL-1 receptor antagonists, IL-4, IL-10, IL-11, IL-13, transforming growth factor-β (TGF-β), RDP-58; soluble cytokine receptors such as soluble TNF receptor p55, soluble TNF receptor p75, soluble IL-1 receptor type 2, soluble High Mobility Group Box 1 protein (HMGB-1) receptor and IL-18 binding protein; and blocking antibodies such as for TNF, IL-6, IL-12, HMGB-1, RAGE (Receptor for Advanced Glycosylation End products) and IL-23;
anti-apoptotic factors such as granulocyte-macrophage colony stimulating factor (GM-CSF), basic fibroblast growth factor (bFGF) and TGF-β;
factors that induce growth arrest such as Dkk-1 and lactoferrin which inhibits GLP-2 mediated epithelial proliferation; and
factors that support the integrity of the mucosal epithelium, including transporters such as transferrines.

In one aspect of the present invention, the factors that ameliorate the symptomatic features associated with mucositis are selected from anyone of the foregoing, in particular from growth factors, blocking antibodies, soluble cytokine receptors and anti-inflammatory cytokines. The growth factors in particular being selected from the group consisting of KGF-1, KGF-2, FGF-20, EGF, GLP-2, R-spondin-1, Insulin-like growth factor, GM-CSF and TGF-beta3; the anti-inflammatory cytokines are in a further embodiment selected from the group consisting of IL-10, IL-13, IL-11, Lactoferrin and RDP-58; the soluble cytokine receptors are in a particular embodiment selected from soluble TNF receptor p75 or soluble HMGB-1 receptor; and the blocking antibodies are in a particular embodiment selected from antibodies or immunogenic binding molecules for IL-6, IL-12, IL-23, RAGE or HMGB-1.

In a first aspect the present invention is based on the finding that recombinant non-pathogenic and non-invasive microorganisms, in particular gram-positive food grade bacterium, such as the lactic acid fermenting bacterial strains, Lactococcus, Lactobacillus or Bifidobacterium species; expressing trefoil peptides are particularly useful in the treatment of oral and intestinal mucositis.

It accordingly provides recombinant non-pathogenic and non-invasive microorganism; in particular gram-positive food grade bacterium, such as the lactic acid fermenting bacterial strains, Lactococcus, Lactobacillus or Bifidobacterium species; more in particular the plasmid free Lactococcus lactis strain MG1363; expressing one or more biologically active polypeptides for use in the prevention or treatment of mucositis; in particular for use in the prevention or treatment of lesions in the mucosal lining of the alimentary tract, especially to the oral, oropharyngeal, intestinal and rectal mucosa.

Given the general understanding that gram-positive bacteria cause significant secondary infections in patients (Dunca M. et al., Alimentary Pharmacology & Therapeutics Vol. 18(9), 2003), it was not to be expected that gram-positive food grade bacteria, would be useful in the prevention/treatment of oral and intestinal mucositis. Also the observation that the use of gram-positive food grade bacteria, enhances the therapeutic effect of trefoil peptides when compared to the art, was beyond expectation.

This in one embodiment, the present invention provides recombinant non-pathogenic and non-invasive microorganism; in particular gram-positive food grade bacterium, such as the lactic acid fermenting bacterial strains, Lactococcus, Lactobacillus or Bifidobacterium species; more in particular the plasmid free Lactococcus lactis strain MG1363; expressing one or more trefoil peptides for use in the prevention or treatment of mucositis; in particular for use in the prevention or treatment of lesions in the mucosal lining of the alimentary tract, especially to the oral, oropharyngeal, intestinal and rectal mucosa. In said embodiment the trefoil peptides particularly consist of TFF1 and/or TFF3; more in particular TFF1 as defined hereinbefore.

In a further aspect the present invention is based on the finding that recombinant non-pathogenic and non-invasive microorganisms expressing adhesive binders enhance the delivery of other factors, such as the factors that target the alimentary tract epithelium mentioned above. We even, surprisingly observed, that the expression of said adhesive binders lead to the delivery of proteins and peptides, even to parts of the alimentary tract such as the oral cavity and upper part of the gut, were the interaction of the adhesive binders to the mucosal wall was not be expected. It is accordingly a further object of the present invention to provide recombinant non-pathogenic and non-invasive microorganisms expressing adhesive binders, optionally further expressing one or more factors desirably to be delivered using said recombinant non-pathogenic and non-invasive microorganism. Alternatively, the adhesive binder expressing microorganisms are used in combination with other recombinant non-invasive and non-pathogenic microorganisms expressing one or more further factor which is desirable to be delivered.

In a particular embodiment, these further factors are factor that targets the alimentary tract epithelium as defined hereinbefore; in particular these further factors are selected from the group consisting of the TFF family, the growth factors EGF, TGF-α, TGF-β, bFGF, HGF, IGF-I, IGF-II, R-spondin1, DKK1; the soluble cytokine receptors HMGB-1 receptor and IL-18 binding protein; and the cytokines IL-1β, IL-10, IL-11, GM-CSF and IL-8; even more in particular these further factors are selected from the group consisting of the TFF family, the growth factors EGF, TGF-α, TGF-β, bFGF, HGF, IGF-I, IGF-II, R-spondin1, DKK1 and the cytokines IL-1β, IL-10, IL-11, GM-CSF and IL-8.

In one or more of the embodiments provided in the examples hereinafter, the further factors are selected from a trefoil peptide or an anti-inflammatory cytokine; more in particular selected from TFF1, TFF2, TFF3 or IL-10; even more in particular selected from TFF1, TFF3 or IL-10.

The adhesive binders as used herein, are meant to describe factors which aid in the adhesion between the drug delivery system, i.e. the recombinant nonpathogenic and non-invasive bacterium according to the invention, such as for example L. lactis, and the alimentary tract, in particular the oral and intestine mucosa. Expression of the adhesive binders will extend the residence time at the site of drug absorption, intensify contact with the mucosa to increase the drug concentration gradient, ensure immediate absorption without dilution or degradation in the luminal fluid, and localize the delivery system at a certain site. Adhesive binders include mucins; MAPA; lectines, such as WGA and TL; fibronectines; bioadhesives, including fibronectin binding proteins, such as for example Protein F; and absorption enhancers. In particular the adhesive binders used in the different embodiments of the present invention, are selected from the group consisting of lectines, fibronectines, fibronectin binding proteins, mucins, MAPA and absorption enhancers.

The factors and adhesive binders as used hereinbefore, are meant to include any mammalian homolog or artificial polypeptide that are substantially identical, i.e. 70%, 75%, 80%, 85%, 87%, 89%, 90%, 92%, 93%, 94%, 96%, 98% or 99% identical to the corresponding human factor, e.g. IL-10 as used herein is meant to include any mammalian homolog or artificial polypeptide that is substantially identical to human IL-10 (SwissProt Accession No. P22301). Preferably, said factors are encoded by a nucleic acid molecule that hybridizes under high stringency conditions to the coding sequence of the corresponding human factor, e.g. IL-10, as use herein, is encoded by a nucleic acid molecule that hybridizes under high stringency conditions to the coding sequence of human IL-10 (GenBank Accession No. M57627.1).

### Sequence Identity

The percentage identity of nucleic acid and polypeptide sequences can be calculated using commercially available algorithms which compare a reference sequence with a query sequence. The following programs (provided by the National Center for Biotechnology Information) may be used to determine homologies/identities: BLAST, gapped BLAST, BLASTN and PSI-BLAST, which may be used with default parameters.

The algorithm GAP (Genetics Computer Group, Madison, Wis.) uses the Needleman and Wunsch algorithm to align two complete sequences that maximizes the number of matches and minimizes the number of gaps. Generally, the default parameters are used, with a gap creation penalty=12 and gap extension penalty=4.

Another method for determining the best overall match between a nucleic acid sequence or a portion thereof, and a query sequence is the use of the FASTDB computer program based on the algorithm of Brutlag et al (Comp. App. Biosci., 6; 237-245 (1990)). The program provides a global sequence alignment. The result of said global sequence alignment is in percent identity. Suitable parameters used in a FASTDB search of a DNA sequence to calculate percent identity are: Matrix=Unitary, k-tuple=4, Mismatch penalty=1, Joining Penalty=30, Randomization Group Length=0, Cutoff Score=1, Gap Penalty=5, Gap Size Penalty=0.05, and Window Size=500 or query sequence length in nucleotide bases, whichever is shorter. Suitable parameters to calculate percent identity and similarity of an amino acid alignment are: Matrix=PAM 150, k-tuple=2, Mismatch Penalty=1, Joining Penalty=20, Randomization Group Length=0, Cutoff Score=1, Gap Penalty=5, Gap Size Penalty=0.05, and Window Size=500 or query sequence length in nucleotide bases, whichever is shorter.

### Formulations

In one embodiment, the present invention provides a recombinant non-pathogenic and non-invasive microorganism expressing the aforementioned trefoil peptide and/or factor(s). In particular expressing a peptide of the TFF family in combination with one or more of the factors that target the alimentary tract epithelium as defined hereinbefore, more in particular selected from the group consisting of the TFF family; the growth factors EGF, TGF-α, TGF-β, bFGF, HGF, IGF-I, IGF-II, R-spondin1, DKK1; the soluble HMGB-1 receptor; the antibodies or immunogenic binding molecules for IL-6, IL-12, IL-23, RAGE or HMGB-1 and the cytokines IL-1β, IL-10, IL-11, GM-CSF and IL-8.

In an aspect of the present invention, the microorganism is a recombinant yeast, in particular any yeast capable of surviving in the mammalian intestine. Alternatively, said yeast has a known probiotic capacity, such as yeast strains selected from kefir, kombucha or dairy products.

In a particular embodiment, said recombinant yeast is selected from the group consisting of Saccharomyces sp., Hansenula sp., Kluyveromyces sp. Schizzosaccharomyces sp. Zygosaccharomyces sp., Pichia sp., Monascus sp., Geothchum sp and Yarrowia sp. More in particular, said yeast is Saccharomyces cerevisiae, even more in particular said yeast is Saccharomyces cerevisiae subspecies boulardii. In one embodiment of the present invention, the recombinant yeast host—vector system is a biologically contained system. Biological containment is known to the person skilled in the art and can be realized by the introduction of an auxotrophic mutation, preferably a suicidal auxotrophic mutation such as the Thy A mutation, or its equivalents. Alternatively, the biological containment can be realised at the level of the plasmid carrying the gene encoding the anti-inflammatory compound. This can be realized, as a non-limiting example, by using an unstable episomal construct, which is lost after a few generations. Several levels of containment, such as plasmid instability and auxotrophy, can be combined to ensure a high level of containment

In another aspect of the present invention, said non-pathogenic and non-invasive microorganism is a food grade bacterial strain, in particular a gram-positive food grade bacterial strain.

In a further aspect of the present invention said gram-positive food grade bacterial strain is a lactic acid fermenting bacterial strain, in particular a Lactococcus, Lactobacillus or Bifidobacterium species. As used herein, Lactococcus or Lactobacillus is not limited to a particular species or subspecies, but meant to include any of the Lactococcus or Lactobacillus species or subspecies, including Lactococcus garvieae, Lactococcus lactis, Lactococcus lactis subsp. cremoris, Lactococcus lactis subsp. hordniae, Lactococcus lactis, Lactococcus lactis subsp. Lactis, Lactococcus piscium, Lactococcus plantarum, Lactococcus raffinolactis, Lactobacillus acetotolerans, Lactobacillus acidophilus, Lactobacillus agilis, Lactobacillus algidus, Lactobacillus alimentarius, Lactobacillus amylolyticus, Lactobacillus amylophilus, Lactobacillus amylovorus, Lactobacillus animalis, Lactobacillus aviarius, Lactobacillus aviarius subsp. araffinosus, Lactobacillus aviarius subsp. aviarius, Lactobacillus bavaricus, Lactobacillus bifermentans, Lactobacillus brevis, Lactobacillus buchneri, Lactobacillus bulgaricus, Lactobacillus carnis, Lactobacillus casei, Lactobacillus casei subsp. alactosus, Lactobacillus casei subsp. casei, Lactobacillus casei subsp. pseudoplantarum, Lactobacillus casei subsp. rhamnosus, Lactobacillus casei subsp. tolerans, Lactobacillus catenaformis, Lactobacillus cellobiosus, Lactobacillus collinoides, Lactobacillus confusus, Lactobacillus coryniformis, Lactobacillus coryniformis subsp. coryniformis, Lactobacillus coryniformis subsp. torquens, Lactobacillus crispatus, Lactobacillus curvatus, Lactobacillus curvatus subsp. curvatus, Lactobacillus curvatus subsp. melibiosus, Lactobacillus delbrueckii, Lactobacillus delbrueckii subsp. bulgaricus, Lactobacillus delbrueckii subsp. delbrueckii, Lactobacillus delbrueckii subsp. lactis, Lactobacillus divergens, Lactobacillus farciminis, Lactobacillus fermentum, Lactobacillus formicalis, Lactobacillus fructivorans, Lactobacillus fructosus, Lactobacillus gallinarum, Lactobacillus gasseri, Lactobacillus graminis, Lactobacillus halotolerans, Lactobacillus hamsteri, Lactobacillus helveticus, Lactobacillus heterohiochii, Lactobacillus hilgardii, Lactobacillus homohiochii, Lactobacillus iners, Lactobacillus intestinalis, Lactobacillus jensenii, Lactobacillus johnsonii, Lactobacillus kandleri, Lactobacillus kefiri, Lactobacillus kefuranofaciens, Lactobacillus kefirgranum, Lactobacillus kunkeei, Lactobacillus lactis, Lactobacillus leichmannii, Lactobacillus lindneri, Lactobacillus malefermentans, Lactobacillus mali, Lactobacillus maltaromicus, Lactobacillus manihotivorans, Lactobacillus minor, Lactobacillus minutus, Lactobacillus mucosae, Lactobacillus murinus, Lactobacillus nagelii, Lactobacillus oris, Lactobacillus panis, Lactobacillus parabuchneri, Lactobacillus paracasei, Lactobacillus paracasei subsp. paracasei, Lactobacillus paracasei subsp. tolerans, Lactobacillus parakefiri, Lactobacillus paralimentarius, Lactobacillus paraplantarum, Lactobacillus pentosus, Lactobacillus perolens, Lactobacillus piscicola, Lactobacillus plantarum, Lactobacillus pontis, Lactobacillus reuteri, Lactobacillus rhamnosus, Lactobacillus rimae, Lactobacillus rogosae, Lactobacillus ruminis, Lactobacillus sakei, Lactobacillus sakei subsp. camosus, Lactobacillus sakei subsp. sakei, Lactobacillus salivarius, Lactobacillus salivarius subsp. salicinius, Lactobacillus salivarius subsp. salivarius, Lactobacillus sanfranciscensis, Lactobacillus sharpeae, Lactobacillus suebicus, Lactobacillus trichodes, Lactobacillus uli, Lactobacillus vaccinostercus, Lactobacillus vaginalis, Lactobacillus viridescens, Lactobacillus vitulinus, Lactobacillus xylosus, Lactobacillus yamanashiensis, Lactobacillus yamanashiensis subsp. mali, Lactobacillus yamanashiensis subsp. Yamanashiensis and Lactobacillus zeae.

In a particular aspect of the present invention, the gram-positive food grade bacterial strain is Lactococcus lactis or any of its subspecies, including Lactococcus lactis subsp. cremoris, Lactococcus lactis subsp. hordniae, Lactococcus lactis and Lactococcus lactis subsp. Lactis. In another aspect of the present invention, the recombinant gram-positive bacterial strains is a biologically contained system, such as the plasmid free Lactococcus lactis strain MG1363, that lost the ability of normal growth and acid production in milk (Gasson, M. J., 1983 J. Bacteriol. 154; 1-9); or the threonine- and pyrimidine-auxotroph derivative L. lactis strains (Sorensen et al. 2000 Appl. Environ. Microbiol. 66; 1253-1258; Glenting et al., 2002 68; 5051-5056).

The bacterial strains as used herein, have been genetically modified so as to produce and secrete a trefoil peptide and one or more of the aforementioned factors that are exogenous to said bacterial strain. The transformed bacterial strains can be produced by any method known in the art for the bacterial expression of recombinant proteins and would typically include cloning of the isolated nucleic acid molecule that encode for said trefoil peptide and/or other factor targeting the gastrointestinal tract epithelium, into an appropriate vector. In such a vector, the polynucleotide encoding the trefoil peptide and/or any one of the aforementioned factors, is operably linked to a control sequence which is capable of providing for the expression of the coding sequence by the bacterial host cell. In particular such a vector is either an expression vector or a chromosomal integration vector, such as for example described in Steidler L. et al., 2003 Nature biotechnology 21(7); 785-789.

It is accordingly a further objective of the present invention to provide a recombinant non-pathogenic and non-invasive microorganism as provided herein, expressing hTFF1 or hTFF3; in particular a stable germline expression of hTFF3 or hTFF1 in the plasmid free Lactococcus lactis strain MG1363.

The term “operably linked” refers to a juxtaposition wherein the components described are in a relationship permitting them to function in their intended manner. A control sequence “operably linked” to a coding sequence is ligated in such a way that expression of the coding sequence is achieved under condition compatible with the control sequences.

Suitable vectors can be chosen or constructed, containing appropriate regulatory sequences, including promoter sequences, terminator fragments, polyadenylation sequences, enhancer sequences, marker genes and other sequences as appropriate. Appropriate bacterial expression vectors are known to the person skilled in the art as described in Nouaille S. et al., 2003 Genetics and Molecular Research 2; 102-111. Said vectors include but are not limited to the lactose phosphotransferase system, optionally linked to the E. coli bacteriophage T7 promoter; the L. lactis nisA promoter system; and vectors comprising promoters regulated by environmental conditions, such as for example the P170 promoter that is only active at low pH.

Systems for cloning and expression of a polypeptide in a variety of different host cells are well known. See, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992. Particular methods for the transformation of the Lactococcus lactis strains are provided in the experimental part hereinafter, but are illustrative of techniques known in the art, and are not intended to be limiting.

### Therapeutic Application

In a further embodiment the present invention provides the use of a recombinant non-pathogenic and non-invasive microorganisms according to the invention, in the treatment or prevention of mucositis; in particular in the treatment or prevention of mucositis that is due to anti-tumour treatment, i.e. causing lesions in the mucosal lining of the alimentary tract, especially to the oral, oropharyngeal, intestinal and rectal mucosa.

In one objective of the present embodiment, the present invention provides the use of the recombinant microorganisms according to the invention, in the treatment or prevention of oral, especially stomatitis and esophagitis” In one embodiment of the present objective, the micro-organisms are selected from food grade bacterial strain such as a Lactococcus, a Lactobacillus species or a Bifidobacterium; in particular the plasmid free Lactococcus lactis strain MG1363; expressing one or more trefoil peptides, preferably TFF1 or TFF3; more preferably expressing TFF1.

Oral mucositis, is characterised clinically by pain, erythema and the formation of deep, diffuse ulcers. It is particularly common in patients undergoing myelotoxic conditioning regimes before bone-marrow transplantation (BMT) or haematopoietic stem-cell transplantation (HSCT), affect in up to 100% patients. Oral mucositis is rated as the most debilitating and troublesome adverse effect of cancer treatment by patients undergoing HSCT or radiotherapy for head and neck cancer. It can cause difficulty with speaking, swallowing and alimentation and significantly impair daily functioning and quality of life.

“Stomatitis” refers to mucositis affecting any surface of the oral pharyngeal and/or laryngeal epithelial surface, unless otherwise specified.

“Esophagitis” refers to mucositis affecting the esophagus.

In a further objective of the present embodiment, the present invention provides the use of the recombinant microorganism according to the invention, in the treatment of rectal and intestinal mucositis, including mucositis of the small and/or large intestine and/or proctitis. Radiation-induced proctitis is a recognized complication of radiotherapy to the pelvis. It is reported to affect some 5% of irradiated patients. In its most severe form, it leads to massive or intractable hemorrhage, which may necessitate repeated transfusions and hospital admissions.

Anti-tumour or anti-neoplastic treatment (i.e. surgical tumor resection, chemotherapy and radiation therapy) is a medical intervention known to be particularly damaging to mucosal epithelial cells, in particular chemotherapy and radiation therapy can lead to severe inflammation, lesions and ulcerations of the mucosal epithelium. It is accordingly an object of the present invention to provide the use of the aforementioned recombinant non-pathogenic and non-invasive microorganisms in the treatment or prevention of mucositis due to chemotherapy and/or radiotherapy.

In one objective of the present embodiment, the microorganisms are used in the manufacture of a medicament for the treatment and/or prevention of mucositis; in particular in the manufacture of a medicament for the treatment and/or prevention of mucositis that is due to antitumour treatment; more in particular in the manufacture of a medicament for the treatment or prevention of mucositis due to chemotherapy and/or radiotherapy. In a further objective of the present embodiment, the present invention provides in the use of the aforementioned non-pathogenic and non-invasive microorganisms in the manufacture of a medicament for the treatment of oral mucositis, including stomatitis and esophagitis. In an even further objective of the present embodiment, the present invention provides the use of the aforementioned non-pathogenic and non-invasive microorganisms in the manufacture of a medicament for the treatment of intestinal mucositis, including mucositis of the small and/or large intestine, and proctitis. It is also an object of the present invention to provide the use of the aforementioned non-pathogenic and non-invasive microorganisms in the manufacture of a medicament for the prevention or treatment of lesions in the mucosal lining of the alimentary tract, especially to the oral, oropharyngeal, intestinal and rectal mucosa.

In view of the utility of the microorganisms according to the invention, there is provided a method for the treatment of an animal, for example, a mammal including humans, suffering from mucositis, which comprises administering an effective amount of a microorganism, in particular a recombinant bacterium or recombinant yeast according to the present invention to an animal in need thereof.

The effective amount of a recombinant microorganism, which is required to achieve a therapeutical effect will be, of course, vary with the factor(s) as defined hereinbefore and/or adhesive binder expressed by said microorganism, the route of administration, the age and condition of the recipient, and the particular disorder or disease being treated. In all aspects of the invention, the daily maintenance dose can be given for a period clinically desirable in the patient, for example from 1 day up to several years (e.g. for the mammal's entire remaining life); for example from about (2 or 3 or 5 days, 1 or 2 weeks, or 1 month) upwards and/or for example up to about (5 years, 1 year, 6 months, 1 month, 1 week, or 3 or 5 days). Administration of the daily maintenance dose for about 3 to about 5 days or for about 1 week to about 1 year is typical. Nevertheless, unit doses should preferably be administered from twice daily to once every two weeks until a therapeutic effect is observed. The microorganisms producing the actor(s) and/or adhesive binder, may be delivered in mono or combination therapy for treatment of the above indicated diseases. Other constituents of the formulation may include preservatives, inorganic salts, acids, bases, buffers, nutrients, vitamins or other pharmaceuticals.

The microorganisms may be delivered in effective amounts per unit dose of at least 10⁴ colony forming units (cfu) to 10¹² cfu per day, in particular between 10⁶ cfu to 10¹² cfu per day, more in particular between 10⁹ cfu to 10¹² cfu per day. In accordance with the method as described in Steidler et al (Science 2000) or through ELISA the factor(s) and/or adhesive binder are secreted to at least 1 ng to 1 μg for 10⁹ cfu. Based thereon, the skilled person in the art can calculate the range of factor(s) and/or adhesive binder secreted at any other dose of cfu.

For said factor(s) and/or adhesive binder, a suitable daily dose would be from 10 fg to 100 μg per day, in particular from 1 pg to 100 μg per day, more in particular from 1 ng to 100 μg per day.

Using the food bacterial strain, i.e. the plasmid free Lactococcus lactis strain MG1363, the trefoil peptides, more in particular TFF1 or TFF3, may be delivered in a dose inducing a low-dose response. Suitable, said factors are delivered in a dose of at least 10 fg to 100 ng per day, preferably between 1 pg and 50 ng per day, more preferably between 100 pg and 50 ng per day, or preferably between 1 ng and 50 ng, or more preferably between long and 50 ng per day, such as for instance, 20 ng, 30 ng, 40 ng per day.

For the treatment of mucositis, in particular for the treatment and/or prevention of oral or intestinal mucositis, including stomatitis, proctitis and esophagitis; more in particular in the treatment and/or prevention of mucositis due to antitumor treatment, including radiation and chemotherapy; the microorganisms of the present invention may advantageously be employed in combination with other agents used in the treatment of mucositis.

Examples of other agents used in the treatment of mucositis are;
Agents which are capable to reduce mucous absorption of the chemotherapy drugs, such as for example cryotherapy, allopurinol, sulglicotide, nucleoside derivatives or pilocarpine;
Radioprotectants such as amifostine (a phosphorthioate), velafermin and nitroxide radioprotectors;
Agents which reduce the change in epithelial proliferation, such as for example beta-carotene, glutamine or silver nitrate;
Anti-inflammatory agents, including non-steroidal anti-inflammatory agents (NSAID's), inflammatory cytokine inhibitors, mast cell inhibitors, and NF-κB inhibitors;
Antimicrobial agents such as for example chlorhexidine, minocycline, amoxicillin, gentamicin, chlortetracycline and oxytetracycline.

Mast cell inhibitors are chemical or biological agents that suppress or inhibit the function of mast cells, or the mediators released by mast cells. For example, mast cell inhibitors can inhibit degranulation, thereby preventing the release of mediators into the extracellular space. Examples of mast cell degranulation inhibitors include picetannol, benzamidines, tenidap, tiacrilast, disodium chromoglycate, lodoxamide ethyl, and lodoxamide tromethamine. Other agents that inhibit mediator release include staurosporine and CGP41251.

Examples of mast cell mediator inhibitors include agents that block the release or secretion of histamine, such as FK-506 and quercetin; antihistamines such as diphenhydramine; and theophylline.

Other mast cell inhibitors include serine protease inhibitors, such as α-1-protease inhibitor; metalloprotease inhibitors; lisofylline; TNFR-FE (available from Immunex, Seattle, Wash.); benzamidine; amiloride; and bis-amidines such as pentamidine and bis (5-amidino-2 benzimidazolyl) methane.

According to the invention, inflammatory cytokine inhibitors can also be used to treat and prevent mucositis. Inflammatory cytokine inhibitors are chemical or biological agents that suppress or inhibit inflammatory cytokines. Such inhibitors include pyridinyl imidazoles, bicyclic imidazoles, oxpentifylline, thalidomide and gabexate mesilate.

Anti-inflammatory agents can be used in combination with inflammatory cytokine and/or mast cell inhibitors to treat and prevent mucositis according to the invention. Examples of anti-inflammatory agents that can be used in the present invention include the non-steroidal anti-inflammatory drugs (NSAID's) flurbiprofen, ibuprofen, sulindac sulfide, and diclofenac. When NSAID's are administered according to the invention, anti-ulcer agents such as ebrotidine can be administered, e.g., to help protect against gastric mucosal damage.

Other anti-inflammatory agents that can be used in the present invention include misoprostil; methylxanthine derivatives, such as caffeine, lisofylline, orpentoxyfylline; benzydamine; naprosin; mediprin; and aspirin.

Another important class of anti-inflammatory agents includes cyclooxygenase (COX) inhibitors, particularly COX-2 inhibitors. COX-2, an inducible enzyme stimulated by growth factors, lipopolysaccharide, and cytokines during inflammation or cell injury, is responsible for the elevated production of prostaglandins during inflammation. COX-2 inhibitors are especially useful where the invention is used to treat mucositis in cancer patients undergoing chemotherapy or radiation therapy, because of the gastrointestinal tolerability of these inhibitors. COX-2 inhibitors that can be used in the invention include celecoxib, nimesulide, meloxicam, piroxicam, flosulide, etodolac, nabumetone, and 1-[(4-methylsulfonyl) phenyl]-3 trifluoromethyl-5-[(4-fluoro) phenyl]pyrazole.

Other useful anti-inflammatory agents include dual cyclooxygenase/lipoxygenase inhibitors, such as 2-acetylthiophene-2-thiazolylhydrazone, and leukotriene formation inhibitors, such as piriprost.

MMP inhibitors include both the antibacterial tetracyclines such as tetracycline HCl, minocycline and doxyocycline, as well as nonantibacterial tetracyclines. Other agents that can be used to treat or prevent mucositis include the nuclear transcription factor kappa-B (NF-κB) activation inhibitors capsaicin and resiniferatoxin.

For the prevention of mucositis, in particular for the prevention of oral or intestinal mucositis, including stomatitis, proctitis and esophagitis; more in particular in the prevention of mucositis due to antitumor treatment, including radiation and chemotherapy; the microorganisms of the present invention may advantageously be employed in combination with other agents used in the treatment of cancer.

The microorganisms of the present invention are particularly useful for the prevention and treatment of oral or intestinal mucositis in patients who are treated with myeloablative chemotherapy or whole body radiation such as with hematopoietic stem cell transplant as a treatment of cancers of hematopoietic origin, e.g. B- and T-cell lymphoma's.

Other agents, known to cause lesions in the mucosal lining of the alimentary tract, especially to the oral, oropharyngeal, intestinal and rectal mucosa, include but are not limited to; Bleomycin, Capecitabine, Cispalitin, Cytarabine, Daunorubicin, Docetaxel, Doxorubicin, Epirubicin, Etoposide, Fluorouracil, Idarubicin, Methotrexate, Paclitaxel, Pemetrexed, and Teniposide.

In particular, docetaxel plus cisplatin and fluorouracil are used as an induction chemotherapy, followed by chemoradiotherapy, in order to reduce the number of cancer cells and make them more susceptible to the chemoradiotherapy.

Said induction chemotherapy is also performed with cisplatin and fluorouracil, followed by chemoradiotherapy.

Thus, in a further aspect, the present invention provides a method to treat cancer in a patient in need of such treatment, said treatment comprising administering an effective amount of the microorganisms according to the invention, and in particular the plasmid free Lactococcus lactis strain MG1363 expressing TFF1 and/or TFF3; in combination with an effective amount of one or more antitumor agent selected from the group consisting of: Bleomycin, Capecitabine, Cispalitin, Cytarabine, Daunorubicin, Docetaxel, Doxorubicin, Epirubicin, Etoposide, Fluorouracil, Idarubicin, Methotrexate, Paclitaxel, Pemetrexed, and Teniposide. It accordingly provides a combination of an effective amount of the microorganisms according to the invention, and in particular the plasmid free Lactococcus lactis strain MG1363 expressing TFF1 and/or TFF3; with an effective amount of one or more antitumor agent selected from the group consisting of: Bleomycin, Capecitabine, Cispalitin, Cytarabine, Daunorubicin, Docetaxel, Doxorubicin, Epirubicin, Etoposide, Fluorouracil, Idarubicin, Methotrexate, Paclitaxel, Pemetrexed, and Teniposide; for use in the treatment of cancer; in particular in the treatment of i) tumours of the head and neck, including squamous-cell carcinoma of the head and the neck, and ii) solid tumors, including colon cancer and breast cancer.

In one embodiment, the method of treating tumours of the head and neck, comprises administering an effective amount of the microorganisms according to the invention, and in particular the plasmid free Lactococcus lactis strain MG1363 expressing TFF1 and/or TFF3 in combination with an effective amount of one or more antitumor agent selected from the group consisting of; docetaxel, cisplatin and fluorouracil. It accordingly provides a combination of an effective amount of the microorganisms according to the invention, and in particular the plasmid free Lactococcus lactis strain MG1363 expressing TFF1 and/or TFF3; with an effective amount of one or more antitumor agent selected from the group consisting of: docetaxel, cisplatin and fluorouracil; for use in the treatment of tumours of the head and neck.

Generally, the treatment of head and neck cancers comprises administering docetaxel, cisplatin and fluorouracil in combination with the plasmid free Lactococcus lactis strain MG1363 expressing TFF1 and/or TFF3.

All the aforementioned combination treatments include, but are not limited to, the use of said chemotherapeutic agents as an induction chemotherapy, followed by chemoradiotherapy. In particular combination therapy, docetaxel plus cisplatin and fluorouracil in combination with the plasmid free Lactococcus lactis strain MG1363 expressing TFF1 and/or TFF3, followed by chemoradiotherapy. More in particular, said combination therapy is with cisplatin and fluorouracil in combination with the plasmid free Lactococcus lactis strain MG1363 expressing TFF1 and/or TFF3, followed by chemoradiotherapy.

The starting doses of the therapeutic agents (e.g., the antitumor agents docetaxel, cisplatin and fluorouracil) to can be adjusted by the skilled clinician in response to toxicity side effects in the patient, such as for example provided in Marshall R. Posner et al., N Eng. J. Med. 357; 17 Oct. 25, 2007; 1705-1715.

For example, in the treatment of head and neck cancers said treatment comprises administering the plasmid free Lactococcus lactis strain MG1363 expressing TFF1 and/or TFF3 in combination with docetaxel, cisplatin and fluorouracil wherein:

(a) said Lactococcus lactis strain is administered in an amount of at least 10⁴ colony forming units (cfu) to 10¹² cfu per day, in particular between 10⁶ cfu to 10¹² cfu per day, more in particular between 10⁹ cfu to 10¹² cfu per day, for each day of the treatment cycle;

The microorganism will be administered in an daily oral formulation, in particular will be topically applied once, twice, three, or six times daily.

(b) said docetaxel is administered in an amount of about 50 mg/m² to about 200 mg/m², in particular of about 50 mg/m² to about 100 mg/m², more in particular about 75 mg/m², once during the treatment cycle;
(c) said cisplatin is administered in an amount of about 50 mg/m² to about 200 mg/m², in particular of about 75 mg/m² to about 150 mg/m², more in particular about 100 mg/m², once during the treatment cycle; and
(d) said fluorouracil is administered in an amount of about 500 mg/m² to about 2000 mg/m², in particular of about 750 mg/m² to about 1500 mg/m², more in particular about 1000 mg/m² per day, for about 3 to 7 days per treatment cycle

A treatment cycle for the methods is, for example, 18 to 28 days. In the methods of this invention, the administration of the microorganisms of the present invention usually starts on day 0, i.e. prior to the administration of the CT/CR, and is administered in an daily oral formulation for each day of the treatment cycle. Docetaxel is usually started on day 1 of the treatment cycle and administered as a intraveneous infusion. cisplatin is usually started on day 1 of the treatment cycle and administered as a intraveneous infusion. Generally, fluorouracil treatment is started on day 1 and administered as a continuous infusion for about 3 to 7 days per treatment.

While it is possible for the bacteria to be administered alone, it is preferable to present it as a composition.

### Compositions

It is also an object of the present invention to provide a composition comprising a non-pathogenic and non-invasive microorganisms; in particular recombinant bacterium or yeast as defined hereinbefore; more in particular recombinant bacterium as defined hereinbefore, even more in particular recombinant gram-positive food grade bacterial strain as defined hereinbefore. In particular, suitable for use in treating and/or preventing mucositis in a subject in need thereof; more in particular for use in treating and/or preventing oral mucositis, including stomatitis and esophagitis, in a subject in need thereof.

The compositions of the present invention, for use in the methods of the present invention, can be prepared in any known or otherwise effective dosage or product form suitable for use in providing topical or systemic delivery of the bacteria to the affected mucosa, which would include both pharmaceutical dosage forms as well as nutritional product forms suitable for use in the methods described herein.

The compositions are preferably administered as oral dosage forms or products that rapidly coat or come in contact with the oral and/or esophageal mucosa, to thus provide more effective contact with the affected mucosal tissue. Preferred dosage or product forms in this respect include mouthwashes which the individual may swish and swallow or swish and spit out. Also preferred are oral lozenges and rectal lavement.

The compositions and methods of the present invention are useful in any pharmaceutical or nutritional liquid product form that can directly or indirectly affect those areas of mucosa which have become or will likely become irritated due to chemical, viral, radiation, or other forms of irritation.

For example, the compositions of the present invention can be formulated in product forms to treat individuals suffering from the mucosal irritation associated with diarrhea or microbial infections such as influenza, rhino viruses, or other microbial infections that can irritate the mucosa.

The pharmaceutical and liquid nutritional product forms are described hereinafter in greater detail.

### Liquid Nutritionals

The compositions of the present invention include liquid nutritional embodiments for oral or enteral administration that comprise one or more nutrients such as fats, carbohydrates, proteins, vitamins, and minerals. Oral liquid nutritionals are preferred.

These nutritional liquids are preferably formulated with sufficient viscosity, flow, or other physical or chemical characteristics to provide a more effective and soothing coating of the affected mucosa while drinking or administering the nutritional liquid. These nutritional embodiments also preferably represent a balanced nutritional source suitable for meeting the sole, primary, or supplemental nutrition needs of the individual.

Non-limiting examples of suitable nutritional liquids within which the prenylflavanoids can be formulated, and thus form selected nutritional liquid embodiments of the present invention, are described in U.S. Pat. No. 5,700,782 (Hwang et al.); U.S. Pat. No. 5,869,118 (Morris et al.); and U.S. Pat. No. 5,223,285 (DeMichele et al.), which descriptions are incorporated herein by reference.

Many different sources and types of carbohydrates, lipids, proteins, minerals and vitamins are known and can be used in the nutritional liquid embodiments of the present invention, provided that such nutrients are compatible with the added ingredients in the selected formulation, are safe and effective for their intended use, and do not otherwise unduly impair product performance.

Proteins suitable for use herein can be hydrolyzed, partially hydrolyzed or non-hydrolyzed, and can be derived from any known or otherwise suitable source such as milk (e.g., casein, whey), animal (e.g., meat, fish), cereal (e.g., rice, corn), vegetable (e.g., soy), or combinations thereof.

Fats or lipids suitable for use in the nutritional liquids include, but are not limited to, coconut oil, soy oil, corn oil, olive oil, safflower oil, high oleic safflower oil, MCT oil (medium chain triglycerides), sunflower oil, high oleic sunflower oil, structured triglycerides, palm and palm kernel oils, palm olein, canola oil, marine oils, cottonseed oils, and combinations thereof.

Carbohydrates suitable for use in the nutritional liquids may be simple or complex, lactose-containing or lactose-free, or combinations thereof. Non-limiting examples of suitable carbohydrates include hydrolyzed corn starch, maltodextrin, glucose polymers, sucrose, corn syrup, corn syrup solids, rice-derived carbohydrate, glucose, fructose, lactose, high fructose corn syrup and indigestible oligosaccharides such as fructo-oligosaccharides (FOS), and combinations thereof.

The nutritional liquids may further comprise any of a variety of vitamins, non-limiting examples of which include vitamin A, vitamin D, vitamin E, vitamin K, thiamine, riboflavin, pyridoxine, vitamin B12, niacin, folic acid, pantothenic acid, biotin, vitamin C, choline, inositol, salts and derivatives thereof, and combinations thereof.

The nutritional liquids may further comprise any of a variety of minerals known or otherwise suitable for us in patients at risk of or suffering from mucositis, non-limiting examples of which include calcium, phosphorus, magnesium iron, selenium, manganese, copper, iodine, sodium, potassium, chloride, and combinations thereof.

### Pharmaceutical Compositions

The pharmaceutical compositions of the present invention can be prepared by any known or otherwise effective method for formulating or manufacturing the selected product form. For example, the bacteria can be formulated along with common excipients, diluents, or carriers, and formed into oral tablets, capsules, sprays, mouth washes, lozenges, treated substrates (e.g., oral or topical swabs, pads, or disposable, non-digestible substrate treated with the compositions of the present invention); oral liquids (e.g., suspensions, solutions, emulsions), powders, or any other suitable dosage form.

Non-limiting examples of suitable excipients, diluents, and carriers include: fillers and extenders such as starch, sugars, mannitol, and silicic derivatives; binding agents such as carboxymethyl cellulose and other cellulose derivatives, alginates, gelatin, and polyvinyl pyrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as acetyl alcohol, glycerol monostearate; adsorptive carriers such as kaolin and bentonite; carriers such as propylene glycol and ethyl alcohol, and lubricants such as talc, calcium and magnesium stearate, and solid polyethyl glycols.

The microorganisms and in particular the yeast and bacteria of the present invention can also be formulated as elixirs or solutions for convenient oral or rectal administration or as solutions appropriate for parenteral administration, for instance by intramuscular, subcutaneous or intravenous routes. Additionally, the nucleoside derivatives are also well suited for formulation as a sustained or prolonged release dosage forms, including dosage forms that release active ingredient only or preferably in a particular part of the intestinal tract, preferably over an extended or prolonged period of time to further enhance effectiveness. The coatings, envelopes, and protective matrices in such dosage forms may be made, for example, from polymeric substances or waxes well known in the pharmaceutical arts.

The compositions of the present invention include pharmaceutical dosage forms such as lozenges, troches or pastilles. These are typically discoid-shaped solids containing the active ingredient in a suitably flavored base. The base may be a hard sugar candy, glycerinated gelatin, or the combination of sugar with sufficient mucilage to give it form. Troches are placed in the mouth where they slowly dissolve, liberating the active ingredient for direct contact with the affected mucosa.

The troche embodiments of the present invention can be prepared, for example, by adding water slowly to a mixture of the powdered active, powdered sugar, and a gum until a pliable mass is formed. A 7% acacia powder can be used to provide sufficient adhesiveness to the mass. The mass is rolled out and the troche pieces cut from the flattened mass, or the mass can be rolled into a cylinder and divided. Each cut or divided piece is shaped and allowed to dry, to thus form the troche dosage form.

If the active ingredient is heat labile, it may be made into a lozenge preparation by compression. For example, the granulation step in the preparation is performed in a manner similar to that used for any compressed tablet. The lozenge is made using heavy compression equipment to give a tablet that is harder than usual as it is desirable for the dosage form to dissolve or disintegrate slowly in the mouth. Ingredients are preferably selected to promote slow-dissolving characteristics.

In a particular formulation of the present invention, the microorganisms will be incorporated in a bioadhesive carrier containing pregelatinized starch and cross-linked poly(acrylic acid) to form a bioadhesive tablet and a bioadhesive gel suitable for buccal application (i.e., having prolonged bioadhesion and sustained drug delivery.

### Bioadhesive Tablet

A powder mixture of drug (the recombinant non-pathogenic and non-invasive bacterium according to the invention), bioadhesive polymers (pregelatinized starch and cross-linked poly(acrylic acid) coprocessed via spray drying), sodium stearyl fumarate (lubricant) and silicium dioxide (glidant) is processed into tablets (weight: 100 mg; diameter: 7 mm). The methods for the production of these tablets are well known to the person skilled in the art and has been described before for the successful development of bioadhesive tablets containing various drugs (miconazol, testosterone, fluoride, ciprofloxacin) (Bruschi M. L. and de Freitas O., Drug Development and Industrial Pharmacy, 2005 31:293-310). All materials are commercially available in pharmaceutical grades, except hopein which is synthesized as described in more detail hereinafter.

To optimize the formulation, the drug load in the tablets and the ratio between starch and poly(acrylic acid) will be varied. Based on previous research, the maximum drug load in the coprocessed bioadhesive carrier is about 60% (w/w) and the starch/poly(acrylic acid) ratio can be varied between 75/25 and 95/5 (w/w). During the optimization study the bioadhesive properties of the tablets and the drug release from the tablets are the main evaluation parameters, with the standard tablet properties (hardness, friability) as secondary evaluation criteria.

### Bioadhesive Gel

The bacteria are incorporated into an aqueous dispersion of pregelatinized starch and cross-linked poly(acrylic acid). This polymer dispersion is prepared via a standard procedure using a high shear mixer.

Similar to the tablet, the drug load of the gel and the starch/poly(acrylic acid) ratio need to be optimized in order to obtain a gel having optimal adherence to the esophageal mucosa. For a gel, the concentration of the polymers in the dispersion is an additional variable as it determines the viscosity of the gel, hence its muco-adhesive properties.

The model to screen the bioadhesive properties of polymer dispersions to the mucosa of esophagus has been described in detail by Batchelor et al. (Int. J. Pharm., 238:123-132, 2002).

### Oral Aqueous Compositions

The compositions of the present invention can be formulated as aqueous compositions for oral administration. Examples of aqueous compositions for oral administration include a mouthwash, mouthrinse, a coating for application to the mouth via an applicator, or mouthspray.

Mouthwash formulations are well-known to those skilled in the art. Formulations pertaining to mouthwashes and oral rinses are discussed in detail, for example, in U.S. Pat. No. 6,387,352, U.S. Pat. No. 6,348,187, U.S. Pat. No. 6,171,611, U.S. Pat. No. 6,165,494, U.S. Pat. No. 6,117,417, U.S. Pat. No. 5,993,785, U.S. Pat. No. 5,695,746, U.S. Pat. No. 5,470,561, U.S. Pat. No. 4,919,918, U.S. Patent Appn. 20040076590, U.S. Patent Appn. 20030152530, and U.S. Patent Appn. 20020044910, each of which is herein specifically incorporated by reference into this section of the specification and all other sections of the specification.

Oral aqueous formulations include such normally employed excipients as, for example, pharmaceutical grades of mannitol, lactose, starch, magnesium stearate, sodium saccharine, cellulose, magnesium carbonate and/or the like. These compositions take the form of solutions such as mouthwashes and mouthrinses, further comprising an aqueous carrier such as for example water, alcoholic/aqueous solutions, saline solutions, parenteral vehicles such as sodium chloride, Ringer's dextrose, etc.

Other additives may be present in the compositions of the invention, such as flavouring, sweetening or colouring agents, or preservatives. Mint, such as from peppermint or spearmint, cinnamon, eucalyptus, citrus, cassia, anise and menthol are examples of suitable flavouring agents. Flavouring agents are preferably present in the oral compositions in an amount in the range of from 0 to 3%; preferably up to 2%, such as up to 0.5%, preferably around 0.2%, in the case of liquid compositions.

Sweeteners include artificial or natural sweetening agents, such as sodium saccharin, sucrose, glucose, saccharin, dextrose, levulose, lactose, mannitol, sorbitol, fructose, maltose, xylitol, thaumatin, aspartame, D-tryptophan, dihydrochalcones, acesulfame, and any combinations thereof, which may be present in an amount in the range of from 0 to 2%, preferably up to 1% w/w, such as 0.05 to 0.3% w/w of the oral composition.

Colouring agents are suitable natural or synthetic colours, such as titanium dioxide or CI 42090, or mixtures thereof. Colouring agents are preferably present in the compositions in an amount in the range of from 0 to 3%; preferably up to 0.1%, such as up to 0.05%, preferably around 0.005-0.0005%, in the case of liquid compositions. Of the usual preservatives, sodium benzoate is preferred in concentrations insufficient substantially to alter the pH of the composition, otherwise the amount of buffering agent may need to be adjusted to arrive at the desired pH.

Other optional ingredients of the aqueous oral compositions may include other active agents such as anti-plaque agents and/or antimicrobial agents. Suitable agents include quaternary ammonium compounds such as domiphen bromide, cetyl pyridinium chloride (CPC), phenolic compounds, ethanol, and the preservatives mentioned above. Such active agents may be present in an amount in the range of from 0 to 4% w/w but may be as much as 70%, such as up to 30%, in the case of ethanol. For example, CPC or the like is preferably present up to 2%, such as about 0.05% w/w, especially in liquid compositions of the invention.

Ethanol may comprise as much as 70%, preferably about 0 to 30% w/w in liquid compositions of the invention, such as about 15% w/w in a mouthspray, but preferred compositions of the invention, particularly oral compositions, are those wherein ethanol or any other alcohol is substantially absent. In case of a mouthspray composition, suitable alcohols include but are not limited to denatured ethanol SD37, denatured ethanol SD37A, denatured ethanol SD38B (SD alcohol 38B), and denatured ethanol SD38A-F. A preferred alcohol is SD alcohol 38B.

Other optional ingredients of oral aqueous compositions according to the invention, may include humectants, surfactants (non-ionic, cationic or amphoteric), thickeners, gums and binding agents.

A humectant adds body to the mouthspray formulation and retains moisture in a dentifrice composition. In addition, a humectant helps to prevent microbial deterioration during storage of the formulation. It also assists in maintaining phase stability and provides a way to formulate a transparent or translucent dentifrice. Suitable humectants include glycerine, xylitol, glycerol and glycols such as propylene glycol, which may be present in an amount of up to 50% w/w each, but total humectant is preferably not more than about 60-80% w/w of the composition. For example, liquid compositions may comprise up to about 30% glycerine plus up to about 5%, preferably about 2% w/w xylitol. Surfactants are preferably not anionic and may include polysorbate 20 or cocoamidobetaine or the like in an amount up to about 6%, preferably about 1.5 to 3%, w/w of the composition.

When the oral compositions of the invention are in the form of a mouthspray, it is preferred to include a film forming agent up to about 3% w/w of the oral composition, such as in the range of from 0 to 0.1%, preferably about 0.001 to 0.01%, such as about 0.005% w/w of the oral composition. Suitable film-formers include (in addition to sodium hyaluronate) those sold under the tradename Gantrez.

This invention will be better understood by reference to the Experimental Details that follow, but those skilled in the art will readily appreciate that these are only illustrative of the invention as described more fully in the claims that follow thereafter. Additionally, throughout this application, various publications are cited. The disclosure of these publications is hereby incorporated by reference into this application to describe more fully the state of the art to which this invention pertains.

### EXAMPLES

The following example illustrate the invention. Other embodiments will occur to the person skilled in the art in light of these examples.

### Example 1

It was previously demonstrated that L. lactis secreting mTFF3 accelerates regeneration of the intestinal epithelium in 5-FU-induced mucositis. Here we wanted to evaluate whether co-expression of Protein F, a Streptococcus pyogenes molecule that mediates adhesion to the extracellular matrix by binding fibronectin, has a synergistic effect on TFF3 treatment in an in vivo model of intestinal mucositis.

### Materials & Methods

### 1.1 Bacteria

The L. lactis strain MG1363 is used throughout the study. Stock solutions of all strains are stored in −20° C. in 50% glycerol in GM17. Bacteria are cultured in GM17 medium, i.e. M17 (Difco Laboratories, Detroit, Mich.) supplemented with 0.5% glucose. For intragastric inoculations, stock suspensions will be diluted 1000-fold in fresh GM17 with the appropriate antibiotic and incubated at 30′C. After 16 hours of incubation, bacteria are harvested by centrifugation and 10-fold concentrated in BM9 medium at 2×10⁹ bacteria/100 μl. Each mouse receives 100 μl of this suspension daily by intragastric catheter.

### 1.2 Plasmids

Mouse Tff3 DNA sequences with optimal L. lactis codon usage are designed and synthesized by PCR assembly of 40 mer oligonucleotides. This tff3 sequence, extended with a sequence encoding the Myc tag, is fused to the usp45 secretion signal of the erythromycin resistant pT1NX vector, downstream of the lactococcal promoter P1. MG1363 strains transformed with the plasmid carrying myc-mTFF3 are designated LL-pT1mycTFF3. LL-pTREX, which is MG1363 containing the empty vector pTREX, will serve as control.

Mouse Tff2 DNA sequences are designed and synthesized by PCR assembly of 40 mer oligonucleotides. This tff2 sequence, extended with a sequence encoding the Myc tag, is fused to the usp45 secretion signal of the erythromycin resistant pT1NX vector, downstream of the lactococcal promoter P1. MG1363 strains transformed with the plasmid carrying myc-mTFF2 are designated LL-pT1mycTFF2.

pPTF5 (Hanski and Caparon, 1992) contains the complete proteinF DNA sequence and was used as template for proteinF amplification. The PCR product was cloned in the erythromycin resistant pT1NX vector between the usp45 secretion signal and the SpaX anchor signal, downstream of the lactococcal promoter P1. MG1363 strains transformed with the plasmid carrying proteinF are designated LL-pT1prtFX. Maxisorp petridishes (35 mm, Nunclon™ Δsurface, Nunc) coated with 1 μg/ml human fibronectin (Sigma) are used to evaluate the binding capacity of LL-pT1prtFX. LL-pTREX serves as control. After washing, bacteria were stained with crystal violet and visualized using light microscopy.

### 1.3 Genomic Integration of ProteinF

Protein F sequence was integrated in the L. lactis genome by homologous recombination between chromosomal and plasmid localized PepN. This was done by cloning proteinF in the PepN gene, thereby inactivating this gene. The resulting strain, designated LL AMB178, has a single copy of proteinF, express protein F at the cell membrane of L. lactis, doesn't form coagulates and is capable of binding fibronectin. LL AMB178 transformed with the plasmids pTREX and pT1mycTFF3 are designated LL-pTREX+protF and LL-pT1mycTFF3+protF, respectively.

### 1.4 Mice

Male C57BL/6 mice (8 weeks old) are purchased from Janvier and housed for 2 weeks before the experiment is performed. They are maintained in a specific pathogen-free central animal facility under conventional conditions.

### 1.5 Induction of Mucositis

We used the chemotherapeutic agent 5-fluorouracil (5-FU) to induce GI mucositis. 216-230 mg/kg 5-FU (Sigma, cat no F6627) was injected IP for 3 consecutive days, PBS (Gibco, cat no 14190) serves as control. A stock solution of 6.9-7.8 mg/ml 5-FU was made and 800 μl was injected. In previous experiments, this regimen has demonstrated to induce gastrointestinal damage in the whole GI tract, especially in the distal part of the small intestine.

### 1.6 Experimental Settings

Mice are randomized into different treatment groups and treated twice daily by oral inoculation with 10⁹ cfu. Treatment starts at day 1, immediately followed by 5-FU administration.

### Groups:

1. LL-pTREX (n=10)
2. LL-pT1 mycTFF3 (n=10)
3. LL-pTREX+protF (n=10)
4. LL-pT1mycTFF3+protF (n=11)
5. LL-pT1mycTFF2 (10)
6. BM9 (n=10)
Total number of mice=61
Every treatment group was divided into 2 cages.

Mice are weighed daily, as relative body weight (RBW) is a good parameter for the development of mucositis.

### Results & Discussion

### 1.1 Relative Body Weight

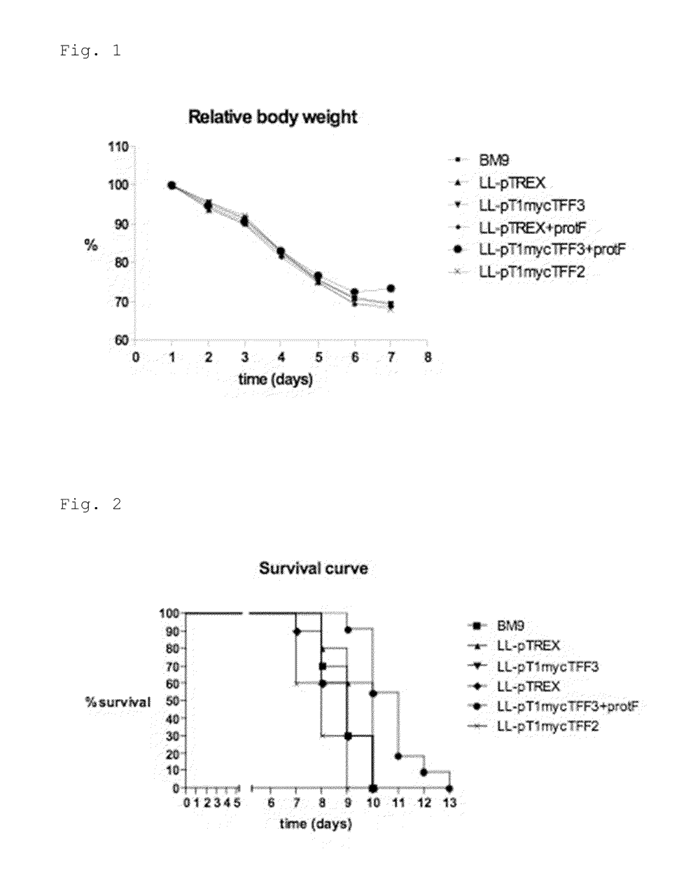

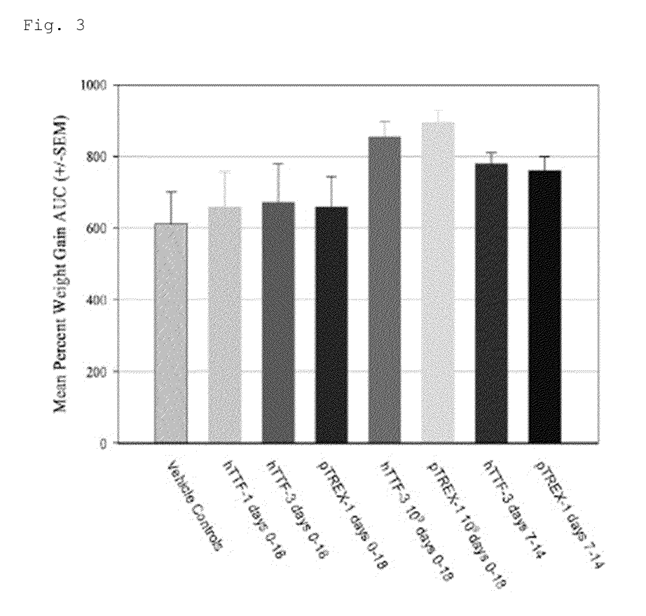

Prior to LL administration and 5-FU injection at day 1, mice are weighed. This weight is used to calculate RBW during the experiment. The RBW is decreasing in all groups, even after a single injection of 5-FU. The RBW of LL-pT1mycTFF3+protF treated mice is significantly higher compared to LL-pTREX+protF (P<0.05). Noticeable, the RBW of LL-pT1mycTFF3+protF treated mice is significantly higher compared to LL-pT1mycTFF2 (P<0.05). (FIG. 1).

### 1.2 Survival

At day 9, almost all LL-pT1mycTFF3+protF treated mice are surviving (91%) while in the other groups, 60% or less survives. The survival rate in pTREX is 60%. The other groups have a survival rate of 30% or lower. At day 10, all mice are dead except 6 mice in the LL-pT1mycTFF3+protF group. (FIG. 2)

### 1.3 Conclusion

5-FU induces body weight loss, even after a single dose of 5-FU. At day 5 and 6, the RBW of LL-pT1mycTFF3+protF treated mice is higher compared to the other groups, but differences are not significant. At day 7, the RBW of LL-pT1mycTFF3+protF treated mice is increasing while the other mice keep losing weight.

MycTFF3 doesn't prevent mortality in mice with 5-FU-induced mucositis, although there is a delay. At day 9, 10 of 11 mice survive in the LL-pT1mycTFF3+protF group while in the control group LL-pTREX+protF, only 30% survives.

In conclusion, binding to fibronectin has a bonus effect on survival in 5-FU-induced mucositis in mice treated with mTFF3 expressing L. lactis strains.

### REFERENCES

Hanski, E. and M. Caparon (1992). “Protein F, a fibronectin-binding protein, is an adhesin of the group A streptococcus Streptococcus pyogenes.” Proc Natl Acad Sci USA 89(13): 6172-6.

### Example 2

Results of earlier studies suggest that TFF3 might be of value in the prevention or treatment of mucositis. The objective of this study was to evaluate the effect of different doses and dosing schedules of TFF1 or TFF3 and expressed by Lactococcus Lactis (LL), on the course of oral mucositis in the acute hamster model. The route of administration was by direct topical application of fresh LL cultures to the hamster cheek pouch.

The acute radiation model used in the present study, has proven to be an accurate, efficient and cost-effective technique to provide a preliminary evaluation of anti-mucositis compounds. Preliminary studies have confirmed the viability and functionality of LL in the hamster cheek pouch, and the activity of TFF expressed in LL.

### Material and Methods

### Study Locations

The study was performed at Biomodels' AAALAC accredited facility in Watertown, Mass. Approval for this study was obtained from the Biomodels Institutional Animal Care and Use Committee (IACUC). The IACUC approval number for this study is 07-0620-01.

### Animals

Male Golden Syrian hamsters (Charles River Laboratories), aged 5 to 6 weeks, with a mean body weight 86 g at study commencement, were used. Animals were individually numbered using an ear punch and housed in groups of 10 animals per cage. Animals were acclimatized for 3 days prior to study commencement. During acclimatization, the animals were observed daily in order to reject animals that presented in poor condition.

### Housing

The study was performed in animal rooms provided with filtered air at a temperature of 70° F.+/−5° F. and 50+/−20% relative humidity. Animal rooms were set to maintain a minimum of 12 to 15 air changes per hour. The room was on an automatic timer for a light-dark cycle of 12 hours on and 12 hours off with no twilight. Sterile Bed-O-Cobs® bedding was used. Bedding was changed a minimum of once per week. Cages, tops, bottles, etc. were washed with a commercial detergent, rinsed and allowed to air dry.

Floors were swept daily and mopped a minimum of twice weekly with a commercial detergent. Walls and cage racks were sponged a minimum of once per month with a dilute bleach solution. The temperature and relative humidity were recorded during the study, and the records retained.

### Diet

Animals were fed with LabDiet® 5061 Rodent Diet and water was provided ad libitum.

### Animal Randomization and Allocations

Hamsters were randomly and prospectively divided into ten groups. Each animal was identified by an ear punch corresponding to an individual number. A cage card identified each cage or label marked with the study number, treatment group number and animal numbers.

### Mucositis Induction

Mucositis was induced using a standardized acute radiation protocol. A single dose of radiation (40 Gy/dose) was administered to all animals on day 0. Radiation was generated with a 160 kilovolt potential (15-ma) source at a focal distance of 50 cm, hardened with a 0.35 mm Cu filtration system. Irradiation targeted the left buccal pouch mucosa at a rate of 3.2 Gy/minute. Prior to irradiation, animals were anesthetized with an intraperitoneal injection of Ketamine (160 mg/ml) and Xylazine (8 mg/ml). The left buccal pouch was everted, fixed and isolated using a lead shield.

### L. Lactis Methods

The growth of L. Lactis strains and the preparation and administration of the cultures followed the protocol below.

### Inoculation of Bacteria:

Bacterial batch cultures (20×1 ml) of LL_hTFF1, LL_hTFF3, LL_PTREX1, sAGX0048, sAGX0057, MG1363 (stored in 100% glycerol in −20° C.) were thaw and briefly vortexed. 100 μl of the bacterial batch cultures was inoculated in a sterile way into 100 ml of GM17E in a sterile 175 ml tube using a disposable 1 ml pipette. These bacterial cultures were stored in a dry incubator at 30° C. until the next morning at 6 AM. The same batch culture was used for the morning and evening inoculation.

Inoculation of sAGX0048, sAGX0057 and MG1363

The bacterial batch cultures (stored in 100% glycerol in −20° C.) were thaw and briefly vortexed. In a sterile way 100 μl of the bacterial batch cultures was inoculated into 105 ml of GM17 in a sterile 175 ml tube using a disposable 1 ml pipette. 5 ml medium was mixed and transferred into a new polystyrene tube. 200 μl of the 100 mM thymidine solution was added to the remaining 100 ml of medium. These bacterial cultures were stored in a dry incubator at 30° C. until the next morning at 6 AM. In the 5 ml sAGX0048 and in sAGX0057 cultures, no bacterial growth should be observed, as these bacteria are thymidine dependent. 100 ml of the bacterial cultures was used to prepare the bacterial suspension. The same batch culture was used for the morning and evening inoculation.

### Preparation of the Bacterial Suspensions

The bacterial cultures of LL_hTFF1, LL_hTFF3 and LL_PTREX1 were spin down (10 min, 21° C., 2900 rpm=1692 g in Eppendorf 5810R), the supernatant discarded and the pellet re-suspended into 2 ml of freshly prepared BM9 (please insert the final concentrations of the ingredients)

The bacterial cultures of sAGX0048, sAGX0057 and MG1363 were spin down (10 min, 21° C., 2900 rpm=1692 g in Eppendorf 5810R), the supernatant discarded and the pellet re-suspended into 2 ml of freshly prepared BM9T (please insert the final concentrations of the ingredients)

### Study Design

Eighty (80) male Syrian Golden Hamsters were given an acute radiation dose of 40 Gy directed to their left buccal pouch. This was accomplished by anesthetizing the animals and everting the left buccal pouches, while protecting the animal body with a lead shield. Test materials were given topically twice daily as detailed in Table 1. At the time of dosing, an aliquot of every culture was retained for quality control purposes.

Mucositis was evaluated clinically starting on day 6, and continuing on alternate days until day 28. At the time of clinical evaluation, the cheek pouches were also photographed. At the end of the study, the photographs were randomized and scored in an independent manner by 2 scorers who were blinded as to the identifiers for each photograph. On day 28, all animals were sacrificed, bled for serum (approximately 300 μl per sample), and the left buccal pouch was excised. Serum was frozen and stored at −70° C. The buccal pouch mucosa was divided into 2 parts. Half of the buccal pouch was fixed in formalin 4% and the other half was snap-frozen in liquid nitrogen, and stored at −80° C.

TABLE 1
Study Design

| | Number of | | | Treatment | | Volume |
|---|---|---|---|---|---|---|
| Group | Animals | Radiation | Treatment | Schedule* | Dosage | (μl) |
| 1 | 10 males | 40 Gy | BM9 vehicle, BID | Day 0 to 18 | vehicle | 100 μl |
| 2 | 10 males | 40 Gy | LL_hTFF1, BID | Day 0 to 18 | 10¹⁰ CFU | 100 μl |
| 3 | 10 males | 40 Gy | LL_hTFF3, BID | Day 0 to 18 | 10¹⁰ CFU | 100 μl |
| 4 | 10 males | 40 Gy | LL_PTREX1, BID | Day 0 to 18 | 10¹⁰ CFU | 100 μl |
| 5 | 10 males | 40 Gy | LL_hTFF3, BID | Day 0 to 18 | 10⁹ CFU | 100 μl |
| 6 | 10 males | 40 Gy | LL_PTREX1, BID | Day 0 to 18 | 10⁹ CFU | 100 μl |
| 7 | 10 males | 40 Gy | LL_hTFF3, BID | Day 7 to 14 | 10¹⁰ CFU | 100 μl |
| 8 | 10 males | 40 Gy | LL_PTREX1, BID | Day 7 to 14 | 10¹⁰ CFU | 100 μl |

### Outcome Evaluation

Study endpoints were mucositis score, weight change and survival. In addition, the plasma and tissue samples taken in this study provide the option for later evaluation by histology, histochemistry, ELISA, PCR or other appropriate technique.

### Survival and Weight Change Data

Animal deaths were evaluated during the course of the study. In general, deaths are usually attributable to adverse effects associated with anesthesia which typically occur at the time of radiation, or toxicity of the experimental compound. Since weight change is a secondary method to examine potential toxicities of experimental treatments, animals were weighed daily throughout the study.

### Mucositis Evaluation

To evaluate mucositis severity, animals were anesthetized with an inhalation anesthetic, and the left cheek pouch everted. Mucositis was scored visually by comparison to a validated photographic scale. The scale ranges from 0 for normal, to 5 for severe ulceration. In descriptive terms, this scale is defined as follows:

TABLE 2
Mucositis Scoring Table

| Score: | Description: |
|---|---|
| 0 | Pouch completely healthy. No erythema or vasodilation |
| 1 | Light to severe erythema and vasodilation. No erosion of mucosa |
| 2 | Severe erythema and vasodilation. Erosion of superficial aspects |
| | of mucosa leaving denuded areas. Decreased stippling of |
| | mucosa. |
| 3 | Formation of off-white ulcers in one or more places. Ulcers may |
| | have a yellow/gray due to pseudomembrane. Cumulative size of |
| | ulcers should equal about ¼ of the pouch. Severe erythema and |
| | vasodilation. |
| 4 | Cumulative size of ulcers should equal about ½ of the pouch. |
| | Loss of pliability. Severe erythema and vasodilation. |
| 5 | Virtually all of pouch is ulcerated. Loss of pliability |
| | (pouch can only partially be extracted from mouth. |

A score of 1-2 is considered to represent a mild stage of the disease, whereas a score of 3-5 is considered to indicate moderate to severe mucositis.

### Results

### Survival and Weight Change Data

No deaths occurred during this study. The mean daily percent weight gains for each group were monitored throughout the study. To evaluate the differences in weight gains between the groups in this study, the mean area under the curve (AUC) was calculated for each animal from the percent weight gain data. Using a one way ANOVA no statistically significant difference in weight change was observed (P=0.055) among any groups in this study (FIG. 3).

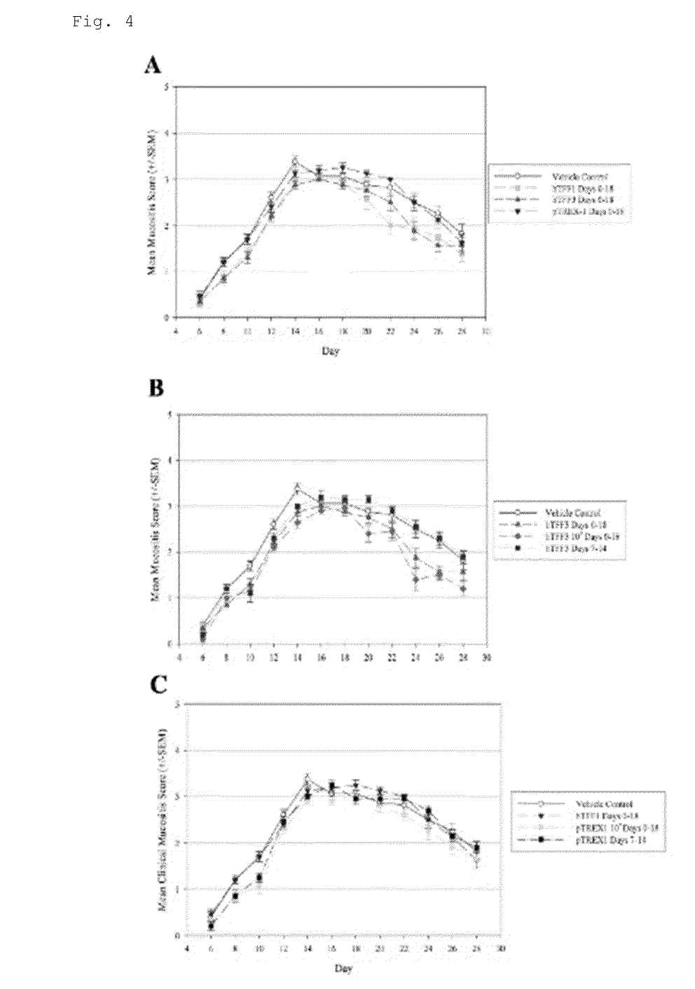

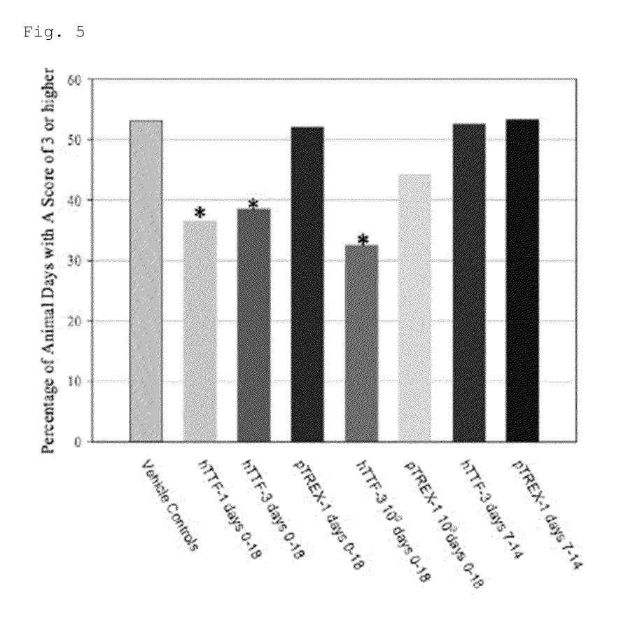

### Mucositis (FIGS. 4 & 5, Table 3)

The mean daily mucositis scores for all study groups are shown in FIG. 4. Topical administration of LL_hTTF1 and LL_hTTF3 favorably altered the course of mucositis. Whereas animals in the radiated vehicle control group demonstrated rapid ulcerative mucositis development from day 10 to a peak of 3.4 on day 14, mucositis development was blunted or abrogated among animals treated with LL_hTFF1 and LL_hTFF3.

### Ulcerative Severity Analysis

The significance of the differences between the vehicle group and the treated groups was assessed in 2 ways, first by the comparison of the number of days with an ulcer (i.e. a score of 3 or higher) using a chi-squared (χ2) test. The results of the analysis of animal days with a score of 3 or higher are shown in Table 3 and FIG. 5. The vehicle control group had scores of 3 or higher on 102 of 192 animal days evaluated (53.1%). There were no significant differences between the irradiated vehicle control group and the groups treated with LL_pTREX-1 at 10¹⁰ bacteria on days 0-19 (P=0.919), days 7-14 (p=0.957), or 10⁹ bacteria on days 0-18 (P=0.079). There was also no significant difference between the group treated with LL_hTFF3 on days 7-14 and the vehicle control group (P=0.974).

TABLE 3
Chi-squared analysis of number of animal days with a mucositis score of 3 or higher.

| | | | | | vs vehicle | |
|---|---|---|---|---|---|---|
| | | | | | Chi Sq | |
| Group | Days >= 3 | Days < 3 | Total Days | % Days >= 3 | v control | P Value |
| Vehicle | 102 | 90 | 192 | 53.1 | | |
| LL_hTFF1 | 70 | 122 | 192 | 38.5 | 10.12000 | 0.001 |
| Day 0 to Day 18 | | | | | | |
| LL_hTFF3 | 74 | 118 | 192 | 38.5 | 7.64700 | 0.006 |
| Day 0 to Day 18 | | | | | | |
| LL_PTREX1 | 100 | 92 | 192 | 52.1 | 0.00104 | 0.919 |
| Day 0 to Day 18 | | | | | | |
| 10⁹ LL_hTFF3 | 78 | 162 | 240 | 32.5 | 17.83000 | <0.001 |
| Day 0 to Day 18 | | | | | | |
| 10⁹ LL_PTREX1 | 106 | 134 | 240 | 44.2 | 3.07900 | 0.079 |
| Day 0 to Day 18 | | | | | | |
| LL_hTFF3 | 126 | 114 | 240 | 52.5 | 0.00104 | 0.974 |
| Day 7 to Day 14 | | | | | | |
| LL_PTREX1 | 128 | 112 | 240 | 53.3 | 0.00291 | 0.957 |
| Day 7 to Day 14 | | | | | | |

To examine the levels of clinically significant mucositis, as defined by presentation with open ulcers (score≧3), the total number of days in which an animal exhibited an elevated score was summed and expressed as a percentage of the total number of days scored for each group. Statistical significance of observed differences was summed and expressed as a percentage of the total number of days scored for each group. Statistical significance of observed differences was calculated using chi-square analysis. Significant improvement is shown Red underline

Both groups treated with LL_hTFF3 from day 0 to day 18 had a significant reduction in the days with a score of 3 or higher, when compared to the vehicle controls, with the lower dose (10⁹) group having less severe mucositis (P<0.001), than the higher dose (10¹⁰) group (P=0.006).

The group treated with LL_hTFF1 at 10¹⁰ bacteria on days 0-18 had a significant reduction in the number of days with severe mucositis to 36.5% (P=0.001)

### Rank Sum Analysis

Further analysis of the data was performed using the Mann-Whitney Rank-sum analysis on the mucositis scores for individual days. In this analysis the data for each group at each time-point is compared with the irradiated vehicle control group. The results of this analysis are shown in Table 4. No significant improvements were seen in the group treated with LL_pTREX1 at 10¹⁰ bacteria/dose on days 0-18.

Two groups had significant improvements in the course of mucositis. The group treated with LL_TFF3 at 10¹⁰ bacteria/dose on days 0-18 showed significant reductions in mucositis scores on days 10 (P=0.049), day 12 (P=0.031) 14 (P=0.029), 24 (P=0.043) and 26 (P=0.012). The group treated with LL_TFF3 at 10⁹ bacteria/dose on days 0-18 showed significant reductions in mucositis scores on days 10 (P=0.003), day 12 (P=0.007) 14 (P=0.003), 24 (P=0.004), 26 (P=0.004), and 28 (P=0.011).

### Example 3

Results of earlier studies suggest that TFF and IL-10 might be of value in the prevention or treatment of mucositis (deKoning B A et al., 2007; Beck P L et al., 2004; deKoning B A et al., 2006). Using the acute hamster model described hereinbefore, it was the objective of the present study to evaluate the effect of different protein constructs of TFF or IL-10 in Lactococcus Lactis (LL) on the course of oral mucositis. The route of administration was by direct topical application of fresh LL cultures of the hamster cheek pouch.

### Material and Methods

But for the study design (infra), the materials and methods used in the present example are almost identical to methods and materials of example 2 above. In said methods the present example differs in that the animals had an average weight of 84 g at study commencement, and the mucositis induction was done using a 250 kilovolt potential (15-ma) source.

### Study Design

One hundred (100) male Syrian Golden Hamsters were given an acute radiation dose of 40 Gy directed to their left buccal pouch. This was accomplished by anesthetizing the animals and everting the left buccal pouches, while protecting the animal body with a lead shield. Test materials were given topically twice daily as detailed in Table 4.

Mucositis was evaluated clinically starting on day 6, and continuing on alternate days until day 28. At the time of clinical evaluation, the cheek pouches were also photographed. At the end of the study, the photographs were randomized and scored in an independent manner by 2 scorers who were blinded as to the identifiers for each photograph. On day 28, all animals were sacrificed, bled for serum (approximately 300 μl per sample), and the left buccal pouch was excised. Serum was frozen and stored at −70° C. The buccal pouch mucosa was divided into 2 parts. Half of the buccal pouch was fixed in formalin 4% and the other half was snap-frozen in liquid nitrogen, and stored at −80° C.

TABLE 4
Study Design

| | Number of | | | Treatment | |
|---|---|---|---|---|---|
| Group | Animals | Radiation | Treatment | Schedule* | Volume (μl) |
| 1 | 10 males | none | BM9 vehicle, BID | Day 0 to 28 | 100 μl |
| 2 | 10 males | 40 Gy | BM9 vehicle, BID | Day 0 to 28 | 100 μl |
| 3 | 10 males | 40 Gy | LL_hIL10, BID | Day −3 to 28 | 100 μl |
| 4 | 10 males | 40 Gy | LLfbp_hIL10, BID | Day −3 to 28 | 100 μl |
| 5 | 10 males | 40 Gy | LL_hTFF2, BID | Day 0 to 28 | 100 μl |
| 6 | 10 males | 40 Gy | LLfbp_hTFF2, BID | Day 0 to 28 | 100 μl |
| 7 | 10 males | 40 Gy | LL_hTFF3, BID | Day 0 to 28 | 100 μl |
| 8 | 10 males | 40 Gy | LLfbp_hTFF3, BID | Day 0 to 28 | 100 μl |
| 9 | 10 males | 40 Gy | LL_PTREX1, BID | Day 0 to 28 | 100 μl |
| 10 | 10 males | 40 Gy | LLfbp_PTREX1, BID | Day 0 to 28 | 100 μl |

*the dose on day 0 is performed 30 minutes prior to radiation. On all other dosing days dosing is done at 6:00 am and 6:00 pm.

### Outcome Evaluation

Study endpoints were mucositis score, weight change and survival were determined as described in example 2. In addition, the plasma and tissue samples taken in this study provide the option for later evaluation by histology, histochemistry, ELISA, PCR or other appropriate technique.

### Results

### Survival

No deaths occurred during this study.

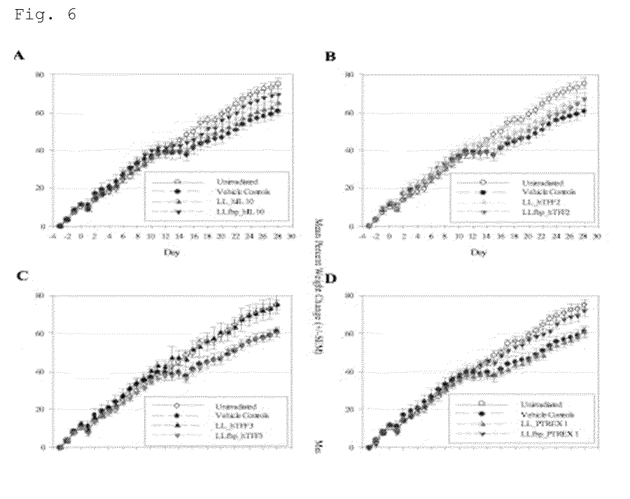

### Weight Change (FIGS. 6 & 7).

The mean daily percent weight gains for each group are shown in FIG. 6. In the unirradiated group the mean percent weight gain during the course of the study was 75.2%. In the vehicle control group the mean percent weight gain during the course of the study was 60.1%. There was a 15% difference in weight gain between the two control groups by day 28. The separation in the weight gain curves between the two control groups (unirradiated vs. placebo) began on day 12 and coincided with the onset of mucositis. This trend was replicated in all groups in which radiation was administered except in animals treated with LL-hTFF3, LL-hIL-10 or LLfbp_PTREX1 in which the weight gain curve was more similar to the unirradiated control group.

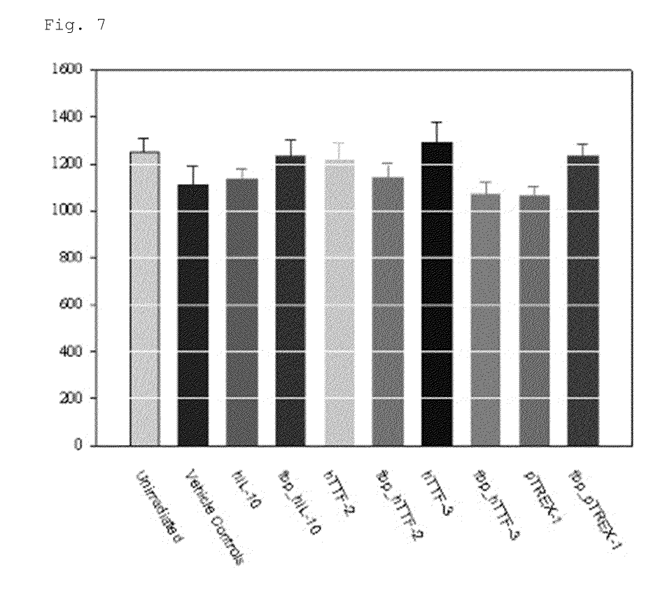

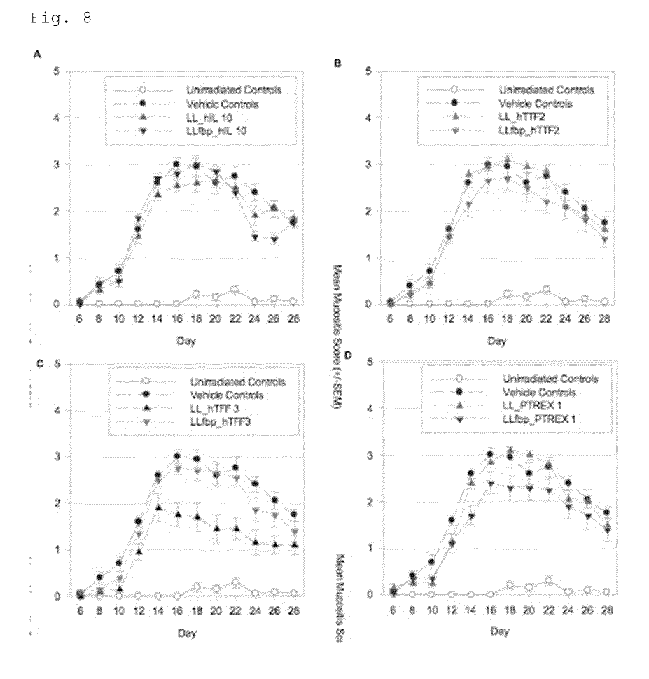

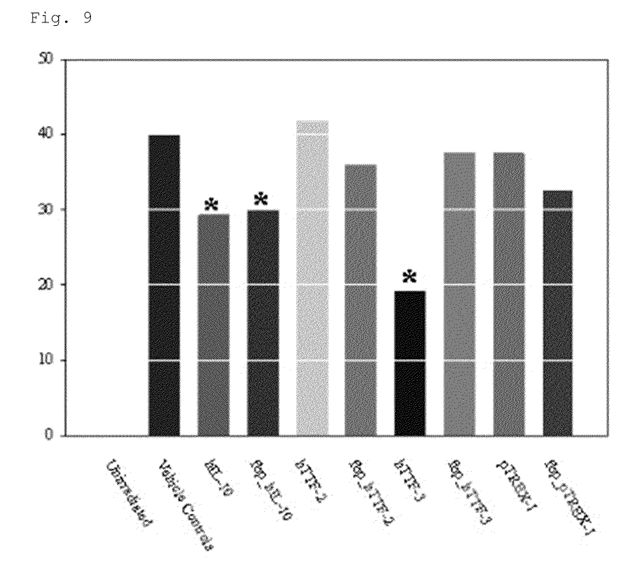

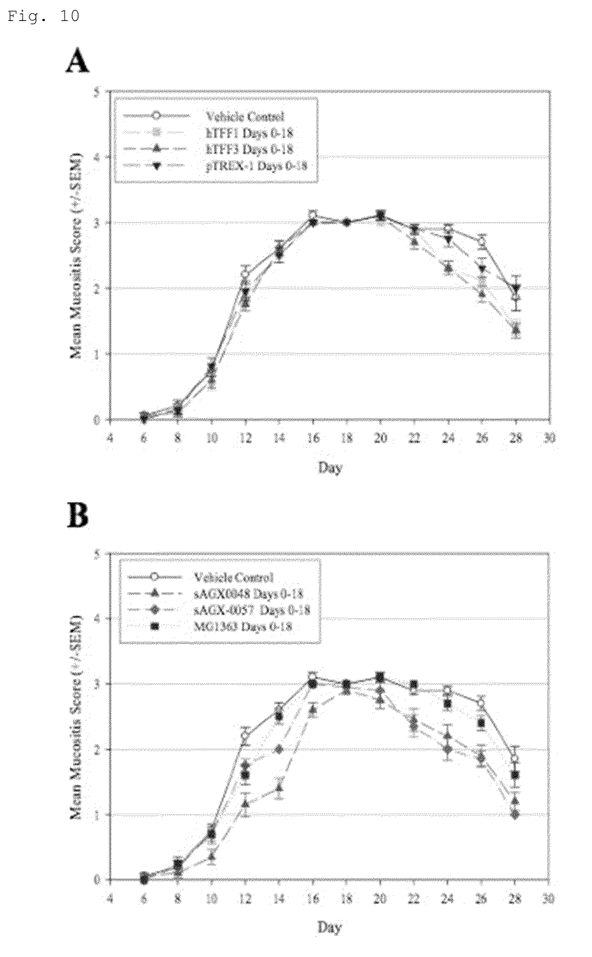

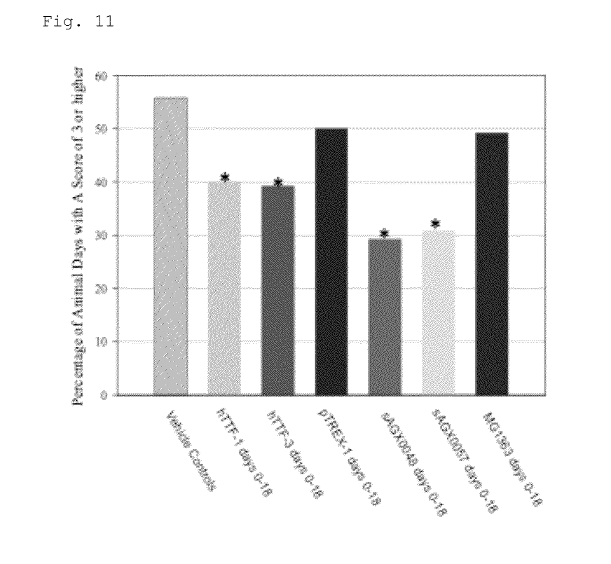

To evaluate the significance of these differences, the mean area under the curve (AUC) was calculated for each animal from the percent weight gain data, and the means and standard errors were plotted (FIG. 7). Using a one way ANOVA no statistically significant difference in weight change was observed (P=0.119) among any groups in this study.

### Mucositis (FIGS. 8 & 9, Table 5)

The mean daily mucositis scores for all study groups are shown in FIG. 8. No mucositis was seen in the unirradiated control group, with few exceptions, all scores in this group were 0. There were several scores of 1 observed in this group. These scores reflect the normal changes observed with this model.

Topical administration of IL-10, TFF, and PTREX1 favorably altered the course of mucositis. Whereas animals in the radiated placebo control group demonstrated rapid ulcerative mucositis development from day 10 to a peak of 3.0 on day 16, mucositis development was blunted or abrogated among animals treated with LL-hIL10, LLfbp_hTFF2, LL_hTFF3 and LLfbp_PTREX1. Of these, it appeared that LL_hTFF3 was most effective. Although there was no consistent impact of the presence of fibronectin, it can not be excluded from the present data that under certain conditions/combinations, fibronectin could have a beneficial effect on mucositis. For example, whereas LL_hTFF3 outperformed LLfbp_hTFF3, LLfbp_PTREX1 and LLfbp_hTFF2 animals did better than those receiving LL_PTREX1 or LL_hTFF2 respectively.

TABLE 5
Chi-squared analysis of number of animal days with a mucositis score of 3 or higher.

| | | | | | vs vehicle | |
|---|---|---|---|---|---|---|
| | | | | | Chi Sq | |
| Group | Days >= 3 | Days < 3 | Total Days | % Days >= 3 | v control | P Value |
| Unirradiated | 0 | 240 | 240 | 0.0 | 117.513 | <0.001 |
| Vehicle | 96 | 144 | 240 | 40.0 | — | — |
| LL_hIL10 | 70 | 170 | 240 | 29.2 | 5.756 | 0.016 |
| LLfbp_hIL10 | 72 | 168 | 240 | 30.0 | 4.844 | 0.028 |
| LL_hTFF2 | 100 | 140 | 240 | 41.7 | 0.078 | 0.781 |
| LLfbp_hTFF2 | 86 | 154 | 240 | 35.8 | 0.717 | 0.397 |
| LL_hTFF3 | 46 | 194 | 240 | 19.2 | 24.012 | <0.001 |
| LLfbp_hTFF3 | 90 | 150 | 240 | 37.5 | 0.2190 | 0.639 |
| LL_PTREX1 | 90 | 150 | 240 | 37.5 | 0.2190 | 0.639 |
| LLfbp_PTREX1 | 78 | 162 | 240 | 32.5 | 2.6050 | 0.107 |

To examine the levels of clinically significant mucositis, as defined by presentation with open ulcers (score ≧3), the total number of days in which an animal exhibited an elevated score was summed and expressed as a percentage of the total number of days scored for each group.
Statistical significance of observed differences was calculated using chi-square analysis.
Significant improvement is shown in Red underline type.

### Ulcerative Severity Analysis

The significance of the differences between the vehicle group and the treated groups was assessed in 2 ways, first by the comparison of the number of days with an ulcer (i.e. a score of 3 or higher) using a chi-squared (χ²) test. The results of the analysis of animal days with a score of 3 or higher are shown in Table 5 and FIG. 9. No scores of 3 or higher were seen in the unirradiated group, while the vehicle control group had scores of 3 or higher on 96 of 240 animal days evaluated (40.0%). There was a statistically significant difference between the unirradiated group and the vehicle control group (P<0.001). There were no significant differences between the irradiated vehicle control group and the groups treated with LL_hTFF2 (P=781), LLfbp_hTFF2 (P=0.397), LLfbp_hTFF3 (P=639), LL_pTREX-1 (P=639), or LLfbp_pTREX-1 (p=0.107).

Both groups treated with IL-10 constructs had significant reductions in mucositis, the group receiving LL_hIL10, had scores of 3 or higher on 70 of 240 animal days evaluated (29.2%, P=0.016), and the receiving LLfbp_hIL10, had scores of 3 or higher on 70 of 240 animal days evaluated (30.0%, P=0.028).

The most significant reduction in the number of animal days with a score of 3 or higher was in the group treated with LL_hTFF3, where a score of 3 or higher was seen on 46 of 240 animal days evaluated (19.2%, P<0.001).

### Rank Sum Analysis

Further analysis of the data was performed using the Mann-Whitney Rank-sum analysis on the mucositis scores for individual days. In this analysis the data for each group at each time-point is compared with the irradiated vehicle control group. The results of this analysis are shown in Table 6. No significant improvements were seen in the groups treated with LL_hTFF2, LLfbp_hTFF2 or LLfbp_hTFF3.

Three groups had significant improvements in the course of mucositis. The group treated with LLfbp_hIL10 showed significant reductions in mucositis scores on days 22 (P=0.013), 24 (P<0.001) and 26 (P=0.008). The group treated with LLfbp_pTREX-1 showed significant reductions in mucositis scores on days 12 (P=0.024), and 14 (P=0.002). The group treated with LL_hTFF3 showed the greatest efficacy of any study group. Mucositis was significantly reduced in 9 evaluation days: days 10 (P=0.012), 12 (P=0.011), 16 (P<0.001), 18 (P=0.006), 20 (P=0.019), 22 (P<0.001), 24 (P=0.001), 26 (P=0.003) and 28 (P=0.016).

TABLE 6
The significance of group differences observed in daily mucositis scores was determined
using the Mann-Whitney rank sum test. This nonparametric statistic is appropriate for the visual
mucositis scoring scale. The p values for each calculation are shown. Significant improvements
are shown in RED

| | Day | | | | | | | | | | | |
|---|---|---|---|---|---|---|---|---|---|---|---|---|
| Group Comparison | 6 | 8 | 10 | 12 | 14 | 16 | 18 | 20 | 22 | 24 | 26 | 28 |
| Vehicle vs | 0.989 | 0.596 | 0.424 | 0.635 | 0.238 | 0.043 | 0.143 | 0.655 | 0.075 | 0.081 | 0.989 | 0.673 |
| LL_hIL10 | | | | | | | | | | | | |
| Day −3 to Day 28 | | | | | | | | | | | | |
| Vehicle vs | 0.795 | 0.797 | 0.424 | 0.178 | 0.596 | 0.259 | 0.673 | 0.694 | 0.013 | <0.001 | 0.008 | 0.849 |
| LLfbp_hIL10 | | | | | | | | | | | | |
| Day −3 to Day 28 | | | | | | | | | | | | |
| Vehicle vs | 0.795 | 0.423 | 0.296 | 0.524 | 0.283 | 0.653 | 0.967 | 0.490 | 0.693 | 0.408 | 0.473 | 0.296 |
| LL_hTFF2 | | | | | | | | | | | | |
| Day 0 to Day 28 | | | | | | | | | | | | |
| Vehicle vs | 0.795 | 0.283 | 0.296 | 0.755 | 0.336 | 0.569 | 0.674 | 0.797 | 0.095 | 0.903 | 0.498 | 0.237 |
| LLfbp_hTFF2 | | | | | | | | | | | | |
| Day 0 to Day 28 | | | | | | | | | | | | |
| Vehicle vs | 0.795 | 0.106 | 0.012 | 0.011 | 0.238 | <0.001 | 0.006 | 0.019 | <0.001 | 0.001 | 0.003 | 0.016 |
| LL_hTFF3 | | | | | | | | | | | | |
| Day 0 to Day 28 | | | | | | | | | | | | |
| Vehicle vs | 0.795 | 0.898 | 0.296 | 0.238 | 0.635 | 0.188 | 0.385 | 0.734 | 0.392 | 0.167 | 0.473 | 0.122 |
| LLfbp_hTFF3 | | | | | | | | | | | | |
| Day 0 to Day 28 | | | | | | | | | | | | |
| Vehicle vs | 0.595 | 0.335 | 0.043 | 0.053 | 0.284 | 0.377 | 0.967 | 0.308 | 0.522 | 0.167 | 0.714 | 0.175 |
| LL_PTREX1 | | | | | | | | | | | | |
| Day 0 to Day 28 | | | | | | | | | | | | |
| Vehicle vs | 0.989 | 0.796 | 0.125 | 0.024 | 0.002 | 0.089 | 0.067 | 0.560 | 0.106 | 0.323 | 0.440 | 0.350 |
| LLfbp_PTREX1 | | | | | | | | | | | | |
| Day 0 to Day 28 | | | | | | | | | | | | |

### Example 4

Were the previous example has demonstrated that TFF-1 and TFF-3 are beneficial in the attenuation of mucositis when produced in Lactococcus Lactis and given as live bacterial cultures. The purpose of this study was to repeat the effect of the plasmid driven strain LL_hTFF1 (AGX-02) and examine the effect of the clinical strains sAGX0048 (secreting hTFF1) and sAGX0057 (secreting hTFF3) in the treatment or oral mucositis. In the clinical strains, the DNA encoding the therapeutic protein is integrated into the bacterial chromosome, rather than part of an exogenous piece of DNA.

### Material and Methods

But for the study design (infra), the materials and methods used in the present example are almost identical to methods and materials of example 2 above. In said methods the present example differs in that the animals had an average weight of 93.8 at study commencement:

### Study Design

Seventy (70) male Syrian Golden Hamsters were given an acute radiation dose of 40 Gy directed to their left buccal pouch. This was accomplished by anesthetizing the animals and everting the left buccal pouches, while protecting the animal body with a lead shield. Test materials were given topically twice daily as detailed in Table 5.

Mucositis was evaluated clinically starting on day 6, and continuing on alternate days until day 28. At the time of clinical evaluation, the cheek pouches were also photographed. At the end of the study, the photographs were randomized and scored in an independent manner by 2 scorers who were blinded as to the identifiers for each photograph. On day 28, all animals were sacrificed, and the left buccal pouch was excised. The buccal pouch mucosa was divided into 2 parts. Half of the buccal pouch was fixed in formalin 4% and the other half was snap-frozen in liquid nitrogen, and stored at −80° C.

TABLE 5
Study Design

| | | | | Treatment | |
|---|---|---|---|---|---|
| Group | Number of Animals | Radiation | Treatment | Schedule* | Volume (μl) |
| 1 | 10 males | none | BM9 vehicle, BID | Day 0 to 28 | 100 μl |
| 2 | 10 males | 40 Gy | LL_hTFF1, BID | Day 0 to 28 | 100 μl |
| 3 | 10 males | 40 Gy | LL_hTFF3, BID | Day −3 to 28 | 100 μl |
| 4 | 10 males | 40 Gy | LL_PTREX1, BID | Day −3 to 28 | 100 μl |
| 5 | 10 males | 40 Gy | sAGX0048, BID | Day 0 to 28 | 100 μl |
| 6 | 10 males | 40 Gy | sAGX0057, BID | Day 0 to 28 | 100 μl |
| 7 | 10 males | 40 Gy | MG1363, BID | Day 0 to 28 | 100 μl |

*the dose on day 0 is performed 30 minutes prior to radiation. On all other dosing days dosing is done at 6:00 am and 6:00 pm.

### Outcome Evaluation

Study endpoints were mucositis score, weight change and survival were determined as described in example 2. In addition, the plasma and tissue samples taken in this study provide the option for later evaluation by histology, histochemistry, ELISA, PCR or other appropriate technique.

### Results

### Survival

No deaths occurred during this study.

### Weight Change

In the vehicle control group the mean percent weight gain during the course of the study was 44.9%. In the group dosed with hTFF1 on days 0-18, the mean percent weight gain during the course of the study was 52.1%. In the group dosed with hTFF3 on days 0-18, the mean percent weight gain during the course of the study was 46.2%. In the group dosed with pTREX1 on days 0-18, the mean percent weight gain during the course of the study was 50.1%. In the group dosed with sAGX0048 on days 0-18, the mean percent weight gain during the course of the study was 45.5%. In the group dosed with sAGX0057 on days 0-18, the mean percent weight gain during the course of the study was 49.0%. In the group dosed with MG1363 on days 0-18, the mean percent weight gain during the course of the study was 46.6%.

To evaluate the significance of these differences, the mean area under the curve (AUC) was calculated for each animal from the percent weight gain data, and the means and standard errors were plotted (data not shown). Using a One Way ANOVA no statistically significant difference in weight change was observed (P=0.372) among any groups in this study.

### Mucositis (FIGS. 10 & 11, Table 6)

The mean daily mucositis scores for all study groups are shown in FIG. 10. Topical administration of LL_hTTF1 and LL_hTTF3 favorably altered the course of mucositis. Similarly, topical application of cultures LL_sAGX0048 and LL_sAGX0057 favorably impacted the course of the mucositis. Cultures of LL_pTREX-1 and LL_MG1363 had little effect on the course of mucositis.

### Ulcerative Severity Analysis

The significance of the differences between the vehicle group and the treated groups was assessed in 2 ways, first by the comparison of the number of days with an ulcer (i.e. a score of 3 or higher) using a chi-squared (χ2) test. The results of the analysis of animal days with a score of 3 or higher are shown in Table 6 and FIG. 11. The vehicle control group had scores of 3 or higher on 134 of 240 animal days evaluated (55.8%). There were no significant differences between the irradiated vehicle control group and the groups treated with LL_pTREX-1 on days 0-18 (P=0.235), or MG1363 (P=0.170).

Both the group treated with LL_hTFF1 and the group treated with LL_hTFF3 from day 0 to day 18 had a significant reduction in the days with a score of 3 or higher when compared to the vehicle controls, (P<0.001 for both groups). Similarly, the groups treated with sAGX0048 or sAGX0057 showed significant reductions in the number of animal days with a score of 3 or higher (P<0.00) for both groups.

TABLE 6
Chi-squared analysis of number of animal days with a mucositis score of 3 or
higher. To examine the levels of clinically significant mucositis, as defined by
presentation with open ulcers (score > 3), the total number of days in which an
animal exhibited an elevated score was summed and expressed as a percentage
of the total number of days scored for each group. Statistical significance of
observed differences was calculated using chi-square analysis. Significant
improvement is shown in Underlined type.

| | | | | | vs vehicle | |
|---|---|---|---|---|---|---|
| | | | | | Chi Sq | |
| Group | Days >= 3 | Days < 3 | Total Days | % Days >= 3 | v control | P Value |
| Vehicle | 134 | 106 | 240 | 55.8 | | |
| LL_hTFF1 | 96 | 144 | 240 | 40.0 | 11.422 | <0.001 |
| Day 0 to Day 18 | | | | | | |
| LL_hTFF3 | 94 | 146 | 240 | 39.2 | 12.707 | <0.001 |
| Day 0 to Day 18 | | | | | | |
| LL_PTREX1 | 120 | 120 | 240 | 50.0 | 1.413 | 0.235 |
| Day 0 to Day 18 | | | | | | |
| sAGX0048 | 70 | 170 | 240 | 29.2 | 33.836 | <0.001 |
| Day 0 to Day 18 | | | | | | |
| sAGX0057 | 74 | 166 | 240 | 30.8 | 29.533 | <0.001 |
| Day 0 to Day 18 | | | | | | |
| MG1363 | 118 | 122 | 240 | 49.2 | 1.880 | 0.170 |
| Day 0 to Day 18 | | | | | | |

### Rank Sum Analysis

Further analysis of the data was performed using the Mann-Whitney Rank-sum analysis on the mucositis scores for individual days. In this analysis the data for each group at each time-point is compared with the irradiated vehicle control group. The results of this analysis are shown in Table 6. No significant improvements were seen in the group treated with LL_pTREX1 on days 0-18. A single day of improved scores were observed in the group treated with MG1363 (P=0.013 on day 12).

The group treated with LL_TFF1 had significantly lower mucositis scores on days 24 (P=0.001) and 26 (P=0.001). The group treated with LL_TFF3 had significantly lower mucositis scores on days 12 (P=0.043), 24 (P=0.001) and 26 (P<0.001). The group treated with sAGX0048 had significantly lower mucositis scores on days 10 (P=0.031), 12 (P<0.001), 14 (P<0.001), 16 (P=0.013), 24 (P=0.005), 26 (P=0.001) and 28 (P=0.038). The group treated with sAGX0057 had significantly lower mucositis scores on days 12 (P=0.043), 14 (P=0.001), 22 (P=0.025), 24 (P<0.001), 26 (P<0.001) and 28 (P=0.003).

TABLE 7
The significance of group differences observed in daily mucositis scores was
determined using the Mann-Whitney rank sum test. This nonparametric statistic
is appropriate for the visual mucositis scoring scale. The p values for each
calculation are shown. Significant improvements are shown in Underline

| | Day | | | | | | | | | | | |
|---|---|---|---|---|---|---|---|---|---|---|---|---|
| Group Comparison | 6 | 8 | 10 | 12 | 14 | 16 | 18 | 20 | 22 | 24 | 26 | 28 |
| Vehicle vs | 0.795 | 0.796 | 0.283 | 0.100 | 0.297 | 0.594 | 0.989 | 0.594 | 0.989 | 0.001 | 0.001 | 0.147 |
| LL_hTFF1 | | | | | | | | | | | | |
| Day 0 to Day 18 | | | | | | | | | | | | |
| Vehicle vs | 0.989 | 0.595 | 0.385 | 0.043 | 0.989 | 0.594 | 0.989 | 0.989 | 0.232 | 0.001 | <0.001 | 0.101 |
| LL_hTFF3 | | | | | | | | | | | | |
| Day 0 to Day 18 | | | | | | | | | | | | |
| Vehicle vs | 0.795 | 0.796 | 0.903 | 0.296 | 0.597 | 0.594 | 0.989 | 0.989 | 0.989 | 0.407 | 0.076 | 0.479 |
| LL_PTREX1 | | | | | | | | | | | | |
| Day 0 to Day 18 | | | | | | | | | | | | |
| Vehicle vs | 0.989 | 0.595 | 0.031 | <0.001 | <0.001 | 0.013 | 0.594 | 0.130 | 0.089 | 0.005 | 0.001 | 0.038 |
| sAGX0048 | | | | | | | | | | | | |
| Day 0 to Day 18 | | | | | | | | | | | | |
| Vehicle vs | 0.795 | 0.989 | 0.796 | 0.043 | 0.001 | 0.594 | 0.795 | 0.633 | 0.025 | <0.001 | <0.001 | 0.003 |
| sAGX0057 | | | | | | | | | | | | |
| Day 0 to Day 18 | | | | | | | | | | | | |
| Vehicle vs | 0.795 | 0.796 | 0.694 | 0.013 | 0.597 | 0.594 | 0.989 | 0.989 | 0.594 | 0.282 | 0.106 | 0.393 |
| MG1363 | | | | | | | | | | | | |
| Day 0 to Day 18 | | | | | | | | | | | | |

### CONCLUSION

Both LL-hTFF1 and LL-hTFF3 applied topically to oral mucosa favorably affected the severity and course of radiation-induced oral mucositis in an established animal model of the condition. Both sAGX0048 and sAGX0057 applied topically to oral mucosa favorably affected the severity and course of radiation-induced oral mucositis in an established animal model of the condition.

The number of days of ulcerative mucositis was not significantly different between the vehicle control group and groups treated with pTREX1 or MG1363.

Based on observed survival and weight changes, the test materials appeared to be well-tolerated.

1. A recombinant non-pathogenic and non-invasive microorganism comprising a food grade bacterial strain selected from the group consisting of a Lactococcus, a Lactobacillus species and a Bifidobacterium, expressing one or more biologically active polypeptides selected from the group consisting of TFF1, TFF2 and TFF3, for use in the treatment of mucositis.

2-4. (canceled)

5. The recombinant non-pathogenic and non-invasive bacterium according to claim 1, wherein said food bacterial strain is a plasmid free Lactococcus lactis strain MG1363.

6-8. (canceled)

9. The recombinant non-pathogenic and non-invasive microorganism according to claim 1, wherein the biologically active polypeptide is a trefoil peptide selected from the group consisting of TFF1 and TFF3.

10. The recombinant non-pathogenic and non-invasive microorganism according to claim 5, expressing hTFF1 or hTFF3.

11-18. (canceled)

19. The recombinant non-pathogenic and non-invasive microorganisms according to according to claim 1, for use in the prevention or treatment of mucositis.

20. A medicament for the prevention or treatment of mucositis comprising a recombinant non-pathogenic and non-invasive microorganisms according to claim 1.

21. The medicament for the prevention or treatment of mucositis according to claim 34, wherein the antitumour treatment is chemotherapy and/or radiotherapy.

22. A medicament for the prevention or treatment of lesions in the mucosal lining of the alimentary tract, comprising the recombinant non-pathogenic and non-invasive bacterium according to claim 1.

23. A medicament for the prevention or treatment of oral mucositis, comprising a recombinant non-pathogenic and non-invasive bacterium according to according to claim 1.

24. A method of treating oral mucositis, comprising administering a recombinant non-pathogenic and non-invasive bacterium according to claim 1, to a mammal, wherein the peptide is delivered to the mammal in a dose of at least 10 fg to 100 ng per day.

25-27. (canceled)

28. A pharmaceutical composition comprising a recombinant non-pathogenic and non-invasive microorganism according to claim 1.

29. The pharmaceutical composition according to claim 28 wherein the composition is an oral dosage form.

30. The pharmaceutical composition according to claim 29, wherein the oral dosage form is selected from the group consisting of; a mouthwash, a mouthspray, an oral lozenge, a bioadhesive tablet and a bioadhesive gel.

31. The recombinant non-pathogenic and non-invasive microorganism according to claim 1, wherein the biologically active polypeptide is a trefoil peptide comprising TFF1.

32. The recombinant non-pathogenic and non-invasive microorganism according to claim 10, expressing a stable germline expression of hTFF3 or hTFF1 in said Lactococcus lactis strain MG1363.

33. The recombinant non-pathogenic and non-invasive microorganisms according to according to claim 19, for use in the prevention or treatment of mucositis that is due to antitumour treatments.

34. The medicament for the prevention or treatment of mucositis of claim 20 comprising recombinant non-pathogenic and non-invasive microorganisms according to according to claim 1, wherein the mucositis is due to antitumor treatments.

35. The medicament for the prevention or treatment of lesions in the mucosal lining of the alimentary tract of claim 22, wherein the treatment of lesions in the mucosal lining of the alimentary tract comprises treatment of lesions in at least one of oral, oropharyngeal, intestinal and rectal mucosa.
